SEQUENCE LISTING (9 sequences)

SEQ ID NO: 1 - DNA, 1743 bp, Homo sapiens
  source 1..1743: mol_type = genomic DNA
atggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcatcgaggaaaaacaggcggagagccggcgggcggtctcggcctgtcaggagatccaggcaatttttacccagaaaagcaagccggggcctgacccgttggacacgagacgcttgcagggctttcggctggaggagtatctgatagggcagtccattggtaagggctgcagtgctgctgtgtatgaagccaccatgcctacattgccccagaacctggaggtgacaaagagcaccgggttgcttccagggagaggcccaggtaccagtgcaccaggagaagggcaggagcgagctccgggggcccctgccttccccttggccatcaagatgatgtggaacatctcggcaggttcctccagcgaagccatcttgaacacaatgagccaggagctggtcccagcgagccgagtggccttggctggggagtatggagcagtcacttacagaaaatccaagagaggtcccaagcaactagcccctcaccccaacatcatccgggttctccgcgccttcacctcttccgtgccgctgctgccaggggccctggtcgactaccctgatgtgctgccctcacgcctccaccctgaaggcctgggccatggccggacgctgttcctcgttatgaagaactatccctgtaccctgcgccagtacctttgtgtgaacacacccagcccccgcctcgccgccatgatgctgctgcagctgctggaaggcgtggaccatctggttcaacagggcatcgcgcacagagacctgaaatccgacaacatccttgtggagctggacccagacggctgcccctggctggtgatcgcagattttggctgctgcctggctgatgagagcatcggcctgcagttgcccttcagcagctggtacgtggatcggggcggaaacggctgtctgatggccccagaggtgtccacggcccgtcctggccccagggcagtgattgactacagcaaggctgatgcctgggcagtgggagccatcgcctatgaaatcttcgggcttgtcaatcccttctacggccagggcaaggcccaccttgaaagccgcagctaccaagaggctcagctacctgcactgcccgagtcagtgcctccagacgtgagacagttggtgagggcactgctccagcgagaggccagcaagagaccatctgcccgagtagccgcaaatgtgcttcatctaagcctctggggtgaacatattctagccctgaagaatctgaagttagacaagatggttggctggctcctccaacaatcggccgccactttgttggccaacaggctcacagagaagtgttgtgtggaaacaaaaatgaagatgctctttctggctaacctggagtgtgaaacgctctgccaggcagccctcctcctctgctcatggagggcagccctg

SEQ ID NO: 2 - DNA, 1743 bp, synthetic construct
  source 1..1743: mol_type = other DNA
atggcggttcgacaagctcttggaagaggcctgcaactgggcagagcactgctgctgagattcaccggaaaacccggcagagcctatggccttggtagacctggacctgccgccggatgtgtcagaggcgaaagacctggatgggctgctggacctggtgccgagcctagaagagttggactgggcctgcctaaccggctgcggttctttagacagtctgtggccggactggccgccagactgcagagacagtttgtcgttagagcctggggctgcgccggaccttgtggtagagctgtgtttctggcctttggcctcggcctgggactgatcgaagagaagcaggccgaaagcagacgggccgtgtctgcctgtcaagagatccaggccatcttcacccagaagtccaagcctggaccagatcctctggacaccagacggctgcagggcttcagactggaagagtacctgatcggccagagcatcggcaagggatgttctgccgccgtgtacgaggccaccatgcctacactgccccagaacctggaagtgaccaagagcacaggactgctgcctggcagaggccctggaacatctgctccaggcgagggacaagaaagagcacctggcgctccagcctttccactggccatcaagatgatgtggaacatcagcgccggcagcagctctgaggccatcctgaataccatgagccaagagctggtgcccgccagcagagttgctctggctggcgaatatggcgccgtgacctaccgcaagtctaagagaggacccaagcagctggcccctcatcctaacatcatccgggtgctgagagccttcaccagcagtgttcctctgcttcctggcgcactggtggactaccctgatgtgcttcctagcagactgcaccctgaaggactcggccacggcagaaccctgttcctcgtgatgaagaactacccctgcacactgcggcagtacctgtgcgtgaacaccccatctccaagactggctgccatgatgctgctgcagctgctggaaggcgtggaccatctggtgcagcagggaatcgcccacagagatctgaagtccgacaacatcctggtggaactggaccccgatggctgtccttggctggttatcgccgatttcggctgctgtctggccgacgagtctatcggactccagctgcctttctcctcttggtacgtggacagaggcggcaacggctgtctgatggctcccgaagtgtctaccgctagaccaggacctagagccgtgatcgactacagcaaagccgatgcctgggccgtgggagccattgcctatgagatcttcggcctggtcaaccccttctacggacaaggcaaagcccacctggaaagcagaagctatcaagaggcccagctgccagctctgcctgagtctgttccacctgatgtgcggcagcttgtcagggctctgctgcagagagaggccagcaaaagaccttctgccagagtggccgccaacgtgctgcatcttagtctgtggggcgagcacattctggccctgaagaacctgaagctggacaagatggtcggatggctgctccagcagtctgccgctacactgctcgccaatagactgaccgagaagtgctgcgtggaaacgaagatgaagatgctgttcctggccaacctggaatgcgagacactgtgtcaggccgctctgctcctctgttcttggagagccgctctg

SEQ ID NO: 3 - DNA, 1743 bp, synthetic construct
  source 1..1743: mol_type = other DNA
atggccgtgcgacaggcactgggccggggcctgcagctggggcgggccctcctgctacggtttaccggcaagcctggcagagcctatggcctgggcagacccggcccagctgccggctgtgtgcggggcgagcgtccaggatgggccgccggacccggggcagagccacgcagagtgggcctgggtctgcccaacaggcttagatttttccgccagagcgtggctggactggctgccaggctgcagcggcagtttgtggtgcgcgcctgggggtgtgccgggccttgcggccgggccgtgtttctcgcctttggactgggactgggcctgattgaagaaaagcaggccgagtccagaagggccgtcagcgcttgccaggagatccaggccattttcacccagaagtcaaagcccggcccagatccactggatacccgaaggctgcagggcttcaggcttgaggagtacctgatcgggcagagtattggcaaaggctgttcagctgccgtgtatgaggcaacaatgcccaccctgccccagaatctggaggtgaccaaatcaacaggactgctgccagggagagggccaggaaccagcgcacccggggagggccaagaacgggcccccggcgcaccagccttccctctggcaatcaagatgatgtggaatatctctgccggcagtagctcagaagccatcctgaatactatgagccaggagctggtccccgcttccagagtggccctggctggcgaatacggcgctgtgacatacaggaagagtaaaaggggcccaaagcagctggcccctcatcccaatataatcagagtgctgagggcctttacctccagcgtgcctttattgcctggcgccctggttgattaccccgacgtgctgccctcacgcctccatcctgagggactgggacatggcaggaccctgtttctggtgatgaagaactatccttgcaccctgcggcagtatttgtgcgtcaatactccatccccccggctggccgccatgatgctgctgcagctcctggaaggagtggatcatctggtgcagcagggaatcgcccatagggacctgaaatccgataacatcctggtggaactggaccccgacggatgtccatggctggtgatcgctgacttcggatgctgcctggccgacgagagcatcggcctgcagctgcctttcagctcttggtatgtagacagaggcggcaatggttgcctgatggcccctgaagtgtctacagcacggcccggacccagggcagtgattgattactccaaggctgatgcatgggccgtgggcgcaatcgcctacgaaatctttgggttggtgaacccattctatggacagggcaaagcccacctggaaagcaggagttaccaggaagcacagctgcccgctctgcctgaatccgtgccacctgatgtgcggcagcttgtgcgggccctgctccaacgcgaagcctctaagagaccatctgcaagagtcgccgccaacgtgctgcatctgagcctgtggggcgaacacatcctggcactgaagaacctgaagctggataagatggtgggatggctgctgcagcagagcgccgccaccctgctggccaaccgtctgaccgaaaaatgctgtgtggagacaaaaatgaagatgctgttcctggctaatctggagtgcgagacactgtgccaggccgccctgctgctgtgcagctggagggccgccctc

SEQ ID NO: 4 - DNA, 469 bp, synthetic construct
  source 1..469: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 5 - DNA, 1730 bp, synthetic construct
  source 1..1730: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 6 - DNA, 1743 bp, synthetic construct
  source 1..1743: mol_type = other DNA
atggccgtgcggcaggccctgggccggggcctgcagctgggcagagccctgctgctgcggttcaccggcaaacctggcagagcctacggcctgggcagacctggcccagccgccggctgcgtgaggggagaaagacccggatgggctgccggcccaggagccgagcctagaagggtgggcctgggcctgcctaacagactgcggttcttccggcaatccgtggccggactcgccgctagactgcagagacaatttgtggtgcgggcctggggatgcgccggcccttgcggccgggccgtgttcctggcttttggcctgggactgggcctgatcgaggaaaagcaggcagaatcccgcagagccgtgtctgcttgccaggagatccaggccatcttcacccagaagagcaaacctggccccgatcctctggacaccagacggctgcagggcttcagactggaagagtacctgattggacagtctatcggcaagggctgtagcgccgctgtctatgaggccacgatgcctacactgccacagaacctggaagtgacaaagagcaccggcctgctgcccggaagaggtcctggaacatctgctcctggcgagggacaggagagagcccctggcgcccctgctttccctctggctatcaagatgatgtggaacatcagcgccggctcctcttctgaagccatcctgaacacaatgagccaggagctggtgcccgccagcagggttgccctggctggagagtacggcgctgtgacatacagaaagtccaagcggggacccaagcagctggccccccaccctaatatcatcagagtgctgagagccttcaccagcagcgtgcctctgctgcctggcgccctggtggactaccctgatgtgctgcctagccggctccatcctgaaggcctgggccacggccggaccctgttcctggtgatgaagaactacccctgcaccctcagacagtacctgtgcgtgaacacccctagccccagactggccgctatgatgctgctgcagctgctggaaggcgtggatcacctggtgcagcagggcatcgcccacagagatctgaagagcgacaacatcctggtggaacttgaccctgacggctgtccttggctggtaatcgccgacttcggctgttgtctggccgacgagagcatcggcctacagctgccattcagctcttggtacgtggacagaggcggcaacggctgcctgatggcccctgaggtgtccaccgccagacctggccccagagccgtgattgactacagcaaggccgatgcctgggccgtgggcgccatcgcctacgagatcttcggcctggtcaaccctttctacggccagggcaaggctcacctggaaagcagaagctaccaggaggcccagctgcccgccctgcctgagagtgtgccacctgacgtgcgccaactcgtcagagccctgctgcaaagagaggccagcaaaagacccagcgcccgtgtggccgccaatgtgctgcacctttctctgtggggcgaacacatcctggccctgaaaaacctcaagctggacaagatggtgggctggctgctgcagcaaagcgccgcgaccctgctggctaatagactgaccgagaagtgctgcgtggaaaccaagatgaaaatgctgtttctggccaacctggagtgcgagacactgtgtcaggcagccctgctgctgtgcagctggcgggccgccctt

SEQ ID NO: 7 - DNA, 1743 bp, synthetic construct
  source 1..1743: mol_type = other DNA
atggctgtccgccaagccttagggagagggctgcagctgggcagagcgctcttgctgaggttcaccggaaagcccggacgcgcttacggcctcggaaggccaggtcctgcagcgggttgcgtgcgaggggaacggcctggttgggcagcaggaccaggagccgagccacggagagtgggactcggcctgcctaaccgcctccgcttcttccggcaaagcgtggccggtcttgccgcacggcttcagcgccagttcgtggtccgggcttggggttgtgccggaccttgtggacgggccgtgttcctggccttcggactgggcctgggactaatcgaggaaaagcaggccgaatcccggagagcggtgtcagcgtgccaggaaatccaggccatattcacccagaagtccaagccgggaccagacccactggacactaggcggctgcagggcttccggctcgaggaatacctcatcggccagtccattggcaaaggttgttcggccgccgtgtacgaggccaccatgcccaccctgccgcaaaacctggaagtgaccaagtccactgggcttctgcccggaagaggacccgggacttctgccccgggagaaggacaggagcgggctcctggagcacccgcgtttccgctggccattaagatgatgtggaacatctcggccggttccagctccgaggccatcctgaacacgatgtcccaagagctggtgccggcgtcaagagtcgccttggctggagaatacggcgccgtgacctaccgcaaatccaagcgcggcccgaaacagctggcgccacaccctaacatcatcagagtgctgcgcgcgttcacctcctccgtgcccctgcttcctggcgctctggtggactatccggacgtgctcccgtcgagactgcaccccgaagggttggggcatggacgcaccctgtttctggtcatgaagaactacccctgcactctgcggcagtatctgtgcgtcaacaccccgtcacctcgccttgccgcgatgatgctgctccaactgctggaaggagtggaccacttggtgcagcagggtatcgcccaccgggatctcaagtccgacaacattctggtggagcttgatcccgatggatgcccctggctggtcatcgccgacttcggctgctgcctggctgacgaaagcatcggactccagttgccgttctcgtcttggtacgtggatcggggcggaaatggctgtctgatggcccccgaagtctccactgcacgccctggacctagggccgtgattgactactcgaaggccgatgcatgggccgtcggagccatcgcctacgagattttcggcttggtgaacccgttctacggccaagggaaggcccacctggaaagcaggagctaccaggaggcccaattgcccgcccttcctgagtccgtgccgcctgacgtgcgacagctcgtgcgcgcactcctgcaacgggaggcctcgaagaggccgtcagctagggtggccgcgaacgtgctgcatctgtcgctgtggggggaacacattctggccctgaagaacctcaagctcgacaagatggtcggctggctgctgcagcaatccgcggccactctgctcgccaatcggctgaccgagaagtgctgcgtggaaaccaagatgaagatgctgtttctcgccaacctggagtgcgaaacactgtgccaggctgccctgctgctctgctcctggagagccgcgctg

SEQ ID NO: 8 - DNA, 1743 bp, synthetic construct
  source 1..1743: mol_type = other DNA
atggcggtcaggcaagcactgggtcgaggactgcagctcggtcgggctcttcttttgcggtttaccggtaaacccggtcgggcatacgggctgggacgacccggacctgccgctggatgtgttagaggggagaggccgggttgggcagccggacccggagcagagccaagaagagtgggtctgggactgcccaaccgactcaggtttttcaggcagtctgtagcagggttggcagctaggctgcaaagacaattcgtggtacgggcgtggggatgcgccgggccatgtggtcgggcggtctttttggccttcggtctggggctcggtttgatcgaggagaaacaagctgagtcccgaagagcggtatctgcgtgtcaagagatacaggccatattcacgcaaaaatcaaagccaggtcccgaccctttggacactaggagactgcaaggattccgcctcgaagagtatctgatcggtcaatcaataggaaaaggttgttcagctgctgtgtatgaagctacgatgcctaccctgcctcaaaatctcgaagtaacgaagtcaaccggattgttgccagggcgagggccaggcacttctgcacctggggaagggcaagagagggcaccgggtgcaccggcattccccctcgcaattaaaatgatgtggaacatatctgccggctcttccagcgaggcaatattgaacacaatgtctcaggaacttgtgcccgctagtcgcgtagcacttgccggcgaatatggtgctgtaacttatagaaaaagcaaaagggggccaaagcagctggcgccgcatcccaacatcataagggtcttgcgagcgttcacttcaagcgtgcctcttcttcctggagcgcttgtcgattatcccgatgtcttgccgtcccgattgcacccagagggtctcggtcacggacgaacgctgttcttggtgatgaaaaactacccttgtactctgcgacaatacttgtgtgtcaacacgccctctcctcgattggcagcgatgatgcttctccaacttcttgagggagtggaccatttggtccagcagggaatcgcgcatcgggaccttaagtcagacaacatattggtagaactcgatccggatggttgcccctggctcgtgatcgcggattttgggtgctgcttggcagacgaatcaataggactgcaattgccgttttctagttggtatgtcgataggggtgggaacgggtgcttgatggcacccgaagtgtctacggcgcgacccggcccacgagcagttattgattactcaaaggcggatgcctgggctgtcggcgctattgcatacgaaatcttcggcctcgtgaaccctttctatgggcaaggaaaagcgcaccttgaatccagaagttaccaagaagcgcagttgcctgcgctcccagagagcgtacctccagatgttagacagctcgtacgggcactgcttcagagagaggcgagcaaaagaccatccgcaagggttgccgccaatgtgttgcatctgtccttgtggggagagcatatcttggcccttaagaatctgaagctggataaaatggtaggatggctcttgcaacaatctgcggcaacgcttctggctaataggctgacagaaaaatgctgcgtagagaccaaaatgaagatgttgttcctcgccaaccttgagtgcgaaacgctctgtcaagcggcattgctcttgtgtagctggagagcagctctt

SEQ ID NO: 9 - protein, 581 aa, Homo sapiens
MAVRQALGRGLQLGRALLLRFTGKPGRAYGLGRPGPAAGCVRGERPGWAAGPGAEPRRVGLGLPNRLRFFRQSVAGLAARLQRQFVVRAWGCAGPCGRAVFLAFGLGLGLIEEKQAESRRAVSACQEIQAIFTQKSKPGPDPLDTRRLQGFRLEEYLIGQSIGKGCSAAVYEATMPTLPQNLEVTKSTGLLPGRGPGTSAPGEGQERAPGAPAFPLAIKMMWNISAGSSSEAILNTMSQELVPASRVALAGEYGAVTYRKSKRGPKQLAPHPNIIRVLRAFTSSVPLLPGALVDYPDVLPSRLHPEGLGHGRTLFLVMKNYPCTLRQYLCVNTPSPRLAAMMLLQLLEGVDHLVQQGIAHRDLKSDNILVELDPDGCPWLVIADFGCCLADESIGLQLPFSSWYVDRGGNGCLMAPEVSTARPGPRAVIDYSKADAWAVGAIAYEIFGLVNPFYGQGKAHLESRSYQEAQLPALPESVPPDVRQLVRALLQREASKRPSARVAANVLHLSLWGEHILALKNLKLDKMVGWLLQQSAATLLANRLTEKCCVETKMKMLFLANLECETLCQAALLLCSWRAAL

(19) United States
(12) Patent Application Publication
(10) Pub. No.: US 2024/0342309 A1
(43) Pub. Date: Oct. 17, 2024

(54) GENE THERAPY VECTORS FOR USE IN PARKINSON'S DISEASE

(71) Applicant: Janssen Pharmaceutica NV, Beerse (BE)

(72) Inventors: Krista Spiller, Philadelphia, PA (US); Geena Skariah, Plymouth meeting, PA (US); Jonathan Haldeman, Lansdale, PA (US)

(21) Appl. No.: 18/433,053

(22) Filed: Feb. 5, 2024

Related U.S. Application Data
(60) Provisional application No. 63/443,289, filed on Feb. 3, 2023.

(51) Int. Cl. A61K 48/00; A61K 38/45; A61P 25/16; C12N 15/86

(52) U.S. Cl. CPC A61K 48/005; A61K 38/45; A61P 25/16; C12N 15/86; C12Y 207/11001; C12N 2750/14143

(57) ABSTRACT

Disclosed herein are recombinant gene therapy vectors comprising a PTEN-induced kinase 1 (PINK1) encoding gene that is operatively linked to a promoter and methods of using the recombinant therapy vectors for inhibiting, reducing, or delaying degeneration or death of neurons of a subject.

### CROSS REFERENCE

This application claims the benefit of U.S. Provisional Application 63/443,289, filed on Feb. 3, 2023, which is incorporated by reference herein in its entirety.

### REFERENCE TO SEQUENCE LISTING SUBMITTED ELECTRONICALLY

This application contains a sequence listing, which is submitted electronically. The contents of the electronic sequence listing (PRD4254WOPCT1 Sequence Listing.xml; Size 21,896 bytes; and Date of Creation: Jan. 25, 2024) is herein incorporated by reference in its entirety.

### FIELD OF THE INVENTION

The present invention relates to gene therapy for inhibiting, reducing, or delaying degeneration or death of neurons in a subject.

### BACKGROUND OF THE INVENTION

Parkinson's disease (PD) is a progressive neurodegenerative disorder, in which symptoms progress slowly from minor motor impairment to severe physical disability which is accompanied by cognitive decline or dementia in some patients. Despite a pressing need, there are currently no clinically approved therapies that slow or cure PD. In most patients the cause of disease is unknown, but a combination of factors including mitochondrial stress likely contributes. Mitochondrial stress has been shown to induce a multitude of cellular dysregulations that can cause both neurodegeneration and neuroinflammation and is particularly linked to PD.

Mutations in the PTEN-induced kinase 1 (PINK1) and Parkin RBR E3 ubiquitin-protein ligase (PARKIN) genes were amongst the first to be associated with familial forms of PD. Homozygous and compound heterozygous loss of function mutations in the PINK1 gene cause autosomal recessive early-onset PD. PINK1 signaling is crucial for maintaining mitochondrial homeostasis due to its essential roles in mitochondrial degradation, mobility, size and network maintenance. Of these, the best characterized function for PINK1 is PARKIN-dependent mitophagy. In healthy mitochondria, PINK1, which is located upstream to PARKIN, is constitutively imported into the mitochondria, cleaved by proteases and subsequently further degraded by the ubiquitin/proteasome system. PARKIN remains inactive in the cytosol. Upon damage or mitochondrial dysfunction (mostly due to mitochondrial depolarization), PINK1 will accumulate at the outer mitochondrial membrane (OMM), leading to the recruitment of PARKIN from the cytosol to the OMM where its E3 ligase activity will ubiquitinate mitochondrial proteins leading to mitochondrial degradation. Beyond its essential function in mitophagy, PINK1 is also involved in the regulation of mitochondrial bio-energetics (complex 1 activation), in calcium homeostasis and mitochondrial quality control (mitochondria derived vesicle pathway). At this moment, there are only a few mitochondria-targeting small molecule therapies in the pipeline, probably due to off-target systemic side-effects associated with modulating ubiquitously present mitochondrial proteins. In addition, according to CiteLine, there are currently 3153 ongoing or completed clinical trials worldwide for PD. However, none of these targets PINK1.

There still is a need to develop a therapeutic that can be delivered to the striatum or substantia nigra directly without systemic side-effects and side-effects on non-targeted CNS resident cells.

### SUMMARY OF THE INVENTION

Provided herein are recombinant gene therapy vectors comprising a polynucleotide operatively linked to a promoter, wherein the vector is an adeno-associated virus (AAV) and the polynucleotide encodes PTEN-induced kinase 1 (PINK1) and has at least about 90% identity to SEQ ID NO: 2 or SEQ ID NO: 3.

In one embodiment of the recombinant gene therapy vector, the PINK1 encoding polynucleotide has at least 95% identity to SEQ ID NO: 2 or SEQ ID NO: 3.

In a further embodiment of the recombinant gene therapy vector, the PINK1 encoding polynucleotide has at least 98% identity to SEQ ID NO: 2 or SEQ ID NO: 3.

In a yet further embodiment of the recombinant gene therapy vector, the PINK1 encoding polynucleotide has at least 99% identity to SEQ ID NO: 2 or SEQ ID NO: 3.

In a yet further embodiment of the recombinant gene therapy vector, the PINK1 encoding polynucleotide has a sequence of SEQ ID NO: 2 or SEQ ID NO: 3.

In a yet further embodiment of the gene therapy vector, the vector is a serotype 1 adeno-associated virus (AAV1).

In a yet further embodiment of the gene therapy vector, the promoter is selected from the group consisting of hSYNI (human synapsin), INA (alpha-internexin), NES (nestin), hTH (human tyrosine hydroxylase), FOXA2 (Forkhead box A2), CaMKII (calmodulin-dependent protein kinase II), NSE (neuron-specific enolase), CMV, CAG, UBC, PGK, EFI-alpha, GAPDH, SV40, HBV, chicken beta-actin, and human beta-actin promoters.

In a yet further embodiment of the gene therapy vector, the vector further comprises one or more regulatory elements. In certain embodiments, the one or more regulatory elements are selected from the group consisting of enhancers, introns, poly-A signal sequence, and transcript stabilizing elements. In certain embodiments, the enhancers are selected from the group consisting of CMV enhancer, GAPDH enhancer, β-actin enhancer, and EF1-α enhancer. In certain embodiments, the transcript stabilizing elements are selected from the group consisting of a WPRE (Woodchuck hepatitis virus posttranscriptional regulatory element) sequence, a HPRE (Hepatitis posttranscriptional regulatory element) sequence, a scaffold-attachment region, a 3′ UTR, and a 5′ UTR.

In a yet further embodiment of the gene therapy vector, the vector comprises an expression cassette comprising in 5′ to 3′ order: a) CAG promoter and polynucleotide encoding PINK1; b) CAG promoter, polynucleotide encoding PINK1, and WPRE; c) hTH promoter and polynucleotide encoding PINK1; d) EF1a promoter and polynucleotide encoding PINK1; e) EF1a promoter, polynucleotide encoding PINK1, and WPRE; f) CBA promoter and polynucleotide encoding PINK1; or g) CBA promoter, polynucleotide encoding PINK1, and WPRE.

Further provided herein are host cells comprising the gene therapy vector provided hereabove.

In one embodiment of the host cell, the host cells are selected from the group consisting of HEK293, 293T, HeLa, Vero, and Sf9 cells.

Yet further provided herein are methods for increasing expression of PINK1 in cells in a subject by contacting the cells with the recombinant gene therapy vector provided hereabove.

In one embodiment of the method, the cells are neurons. In certain embodiments, the neurons are primary tyrosine hydroxylase positive neurons.

In a further embodiment of the method, the subject is a human subject. In certain embodiments, the human subject comprises a mutation in PINK1 gene. In certain embodiments, the human subject is suffering or at risk of developing Parkinson's Disease (PD).

In certain embodiments, the PD is an early-onset PD. In certain embodiments, the PD is an early-onset autosomal recessive PD.

Yet further provided herein are methods for inhibiting, reducing, or delaying degeneration or death of neurons of a subject by contacting the neurons with the gene therapy vector of any one of claims 1-12, thereby increasing the expression level of PINK1 in the neurons and inhibiting, reducing, or delaying dysfunction/degeneration or death of the neurons. In certain embodiments, the subject is a human subject. In certain embodiments, the neurons are a primary tyrosine hydroxylase positive neuron.

In one embodiment of the method, MPP+ (1-methyl-4-phenylpyridinium)-induced neurodegeneration is attenuated in the subject.

In a further embodiment of the methods, MPTP (1-methyl-4-phenyl-1,2,3,6-tetrahydropyridine)-induced neuron loss is prevented in the subject.

In a yet further embodiment of the methods, the human subject comprises a mutation in PINK1 gene.

In a yet further embodiment of the methods, the human subject is suffering or at risk of developing Parkinson's Disease (PD). In certain embodiments, the PD is an early-onset PD. In certain embodiments, the PD is an early-onset autosomal recessive PD.

Yet further provided herein are polynucleotides encoding PINK1 and has at least about 90% identity to SEQ ID NO: 2 or SEQ ID NO: 3.

In one embodiment, the polynucleotide has at least 95% identity to SEQ ID NO: 2 or SEQ ID NO: 3.

In a further embodiment, the polynucleotide has at least 98% identity to SEQ ID NO: 2 or SEQ ID NO: 3.

In a yet further embodiment of the polynucleotide, the PINK1 encoding polynucleotide has at least 99% identity to SEQ ID NO: 2 or SEQ ID NO: 3.

In a yet further embodiment of the polynucleotide, the PINK1 encoding polynucleotide has a sequence of SEQ ID NO: 2 or SEQ ID NO: 3.

Yet further provided herein are plasmids comprising the polynucleotide of any one of claims 32-36.

Yet further provided herein are recombinant gene therapy vectors comprising a variant polynucleotide encoding PINK1, wherein a cell transduced with the vector expresses PINK1 at a higher level in compared to the level of PINK1 expressed in a cell of the same type that is transduced with a vector of the same type comprising a wildtype polynucleotide encoding PINK1.

Yet further provided herein are methods for increasing expression of PINK1 in cells in a subject, the method comprising contacting the cells with a recombinant gene therapy vector comprising a variant polynucleotide encoding PINK1, wherein expression of PINK1 in the cells is increased relative to expression of PINK1 in cells of the same type which are contacted under the same conditions with the same amount of a vector of the same type comprising a wildtype polynucleotide encoding PINK1.

Yet further provided herein are methods for inhibiting, reducing, or delaying dysfunction/degeneration or death of neurons in a subject, the method comprising contacting the neurons with a recombinant gene therapy vector comprising a variant polynucleotide encoding PINK1, wherein expression of PINK1 in the neurons is increased relative to expression of PINK1 in neurons of the same type which are contacted under the same conditions with the same amount of a vector of the same type comprising a wildtype polynucleotide encoding PINK1.

In one embodiment of the vectors or methods provided above, the variant polynucleotide is operatively linked to a promoter.

In a further embodiment of the vector or method provided above, each of the variant polynucleotide and the wildtype polynucleotide is operatively linked to a promoter of the same type.

In a yet further embodiment of the vector or method provided above, the vector is an adeno-associated virus (AAV).

In a yet further embodiment of the vector or method provided above, PINK1 is expressed at least about 1.1, 1.2, 1.3, 1.4, 1.5, 1.6, 1.7, 1.8, 1.9, or 2-fold higher by cells or neurons contacted or transduced with the vector comprising the variant polynucleotide encoding PINK1 compared to cells or neurons contacted or transduced with the vector comprising the wildtype polynucleotide encoding PINK1.

In a yet further embodiment of the vector or method provided above, the variant polynucleotide encoding PINK1 has at least about 70%, at least about 80%, at least about 85%, or at least about 90% identity to SEQ ID NO: 2 or SEQ ID NO: 3.

In a yet further embodiment of the vector or method provided above, the cell is a neuron.

In a yet further embodiment of the vector or method provided above, the neuron is selected from the group consisting of cortical neurons, ependymal cells, glutamatergic neurons, gabaergic neurons, dopaminergic neurons, oligodendrocytes, astrocytes, and microglial cells.

Yet further provided herein are methods for inhibiting, reducing, or delaying degeneration or death of neurons in a subject, the method comprising contacting the neurons with an amount of recombinant gene therapy vector comprising a polynucleotide encoding PINK1, wherein the amount of vector is an amount effective to inhibit, reduce, or delay degeneration or death of neurons in the subject.

In one embodiment of the method, the neurons are contacted with an amount of vector corresponding to a multiplicity of infection (MOI) of between about 1 and about 10\hat{ }5.

In a further embodiment of the method, the neurons are contacted with an amount of vector corresponding to a MOI of between about 10 and about 10\hat{ }4.

In a yet further embodiment of the method, the neurons are contacted with an amount of vector corresponding to a MOI of about 1, about 10, about 10\hat{ }1, about 10\hat{ }2, about 10\hat{ }3, about 10\hat{ }4 or about 10\hat{ }5.

In a yet further embodiment of the method, the polynucleotide is operatively linked to a promoter.

In a yet further embodiment of the method, the polynucleotide is a variant polynucleotide.

In a yet further embodiment of the method, the vector is an adeno-associated virus (AAV).

In a yet further embodiment of the method, the variant polynucleotide encoding PINK1 has at least about 70%, at least about 80%, at least about 85%, or at least about 90% identity to SEQ ID NO: 2 or SEQ ID NO: 3.

In a yet further embodiment of the method, the neuron is selected from the group consisting of cortical neurons, ependymal cells, glutamatergic neurons, gabaergic neurons, dopaminergic neurons, oligodendrocytes, astrocytes, and microglial cells.

### BRIEF DESCRIPTION OF DRAWINGS

FIG. 1A is a Western blot showing PINK1 mRNA expression level in PINK1 KO HEK293T cells transduced with AAV1 CIS plasmids containing WT PINK1 (GT65) or PINK1 variants (GT66, TG69, GT72, GT73, GT74, or GT75).

FIG. 1B is a graph showing the relative PINK1 protein level in PINK1 KO HEK293T cells transduced with AAV1 CIS plasmids containing WT PINK1 (GT65) or PINK1 variants.

FIGS. 2A-2B show representative images from HEK293T wildtype and PINK1 knockout cells that were treated with DMSO vehicle or Valinomycin drug before and after overexpressing AAV1-PINK1. FIG. 2A is an immunocytochemistry of pS65-Ub in PINK1 WT and KO HEK293T cells treated with DMSO vehicle or Valinomycin. FIG. 2B a graph showing pS65-Ub readouts in PINK1 WT and KO HEK293T cells overexpressing PINK1 WT or PINK1 variants with or without Valinomycin treatment.

FIGS. 3A-3E are graphs showing the amount of released LDH in primary cortical neurons transduced with AAV1 vectors containing PINK1 variants GT67 (FIG. 3A), GT69 (FIG. 3B), GT74 (FIG. 3C), GT77 (FIG. 3D), or GT79 (FIG. 3E).

FIG. 4 is a graph showing the amount of released LDH in primary cortical neurons transduced with AAV1 vectors containing GT69 or GT74 at indicated MOIs, in comparison with untransduced neurons.

FIG. 5 shows the in vivo expression of human PINK1 in rats transduced with AAV vectors containing PINK1 WT (Panel A), PINK1 variant GT69 (Panel B), or PINK1 variant GT74 (Panel C). Panel D shows human PINK1 mRNA level in untransduced rat. FIGS. 6 and 7 show western blot and qPCR results, respectively, from striatum samples.

FIG. 8A shows dopamine (DA) levels in the striatum of wildtype and PINK1 KO rodents treated with varying amounts of a control and GT65. FIG. 8B shows dopamine (DA) levels in the striatum of wildtype and PINK1 KO rodents treated with varying amounts of a control and GT69.

FIGS. 9A-9B shows levels of tyrosine hydroxylase (TH) in the midbrain of PINK1 KO rodents treated with varying amounts of GT65 and GT69, as measured by mass spectrometry (FIG. 9A) and by western blot (FIG. 9B).

FIGS. 10A-10D are path maps of WT rats, PINK1 KO rats, or PINK1 KO rats transduced with AAV containing WT PINK1 gene (GT65) or variant PINK1 gene (GT69). FIGS. 10E-10G are graphs showing motor skill changes for PINK1 KO rats after transduction with AAV containing WT PINK1 gene (GT65) or variant PINK1 gene (GT69).

FIG. 11 shows in situ hybridization (ISH) and immunohistochemistry (IHC) for hPINK1 mRNA and protein and canine tyrosine hydroxylase (TH) in the brain of a canine administered a single injection to the substantia nigra of AAV1 vector containing GT69.

### DETAILED DESCRIPTION OF THE INVENTION

The invention is described and claimed herebelow. And it is well established that the subject matter described and claimed herein is to be viewed and interpreted at the time of filing this patent application.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes +10% of the recited value. For example, a concentration of 1 mg/ml includes 0.9 mg/ml to 1.1 mg/ml. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2 111.03.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1992)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

In one aspect, the present disclosure provides a recombinant gene therapy vector comprising an expression cassette for expressing PTEN-induced putative kinase 1 (PINK1) protein (SEQ ID NO: 9, as listed below) and the vector is an adeno-associated vector (AAV). In some embodiments, the expression cassette comprises a polynucleotide that encodes PINK1 and is operatively linked to a promoter.

The polynucleotide that encodes PINK1 may comprise a sequence having at least about 70%, about 75%, 80%, about 85%, about 90%, about 95%, about 96%, about 97%, about 98%, or about 99% identity to SEQ ID NO: 1 (which corresponds to nucleotides 92-1834 of human wildtype human PINK1 gene (NM_032409.3)). In some embodiments, the polynucleotide encoding PINK1 also may be a variant polynucleotide derived from modified wildtype human PINK1 gene. In some embodiments, the polynucleotide encoding PINK1 may comprise a sequence having at least about 70%, about 75%, about 80%, about 85%, about 90%, about 95%, about 96%, about 97%, about 98%, or about 99% identity to a sequence selected from SEQ ID NOs: 2-3. In some embodiments, the polynucleotide encoding PINK1 may comprise a sequence selected from SEQ ID NOs: 2-3. In some embodiments, the polynucleotide encoding PINK1 may comprise a sequence of SEQ ID NO: 2. In some embodiments, the polynucleotide encoding PINK1 may comprise a sequence of SEQ ID NO: 3.

As used herein, “variant polynucleotide encoding PINK1”, “variant PINK1 gene”, and “PINK1 variant” are used interchangeably and to refer to a polynucleotide sequence derived from modified wildtype human PINK1 gene. In other words, the “variant polynucleotide encoding PINK1”, “variant PINK1 gene”, or “PINK1 variant” contains at least one nucleotide change compared to the wildtype human PINK1 gene (SEQ ID NO: 1) and encodes the PINK1 protein (SEQ ID NO: 9).

SEQ ID NO: 1
ATGGCGGTGCGACAGGCGCTGGGCCGCGGCCTGCAGCTGGGTCGAGCGCTGCTGCTGCGCTTC
ACGGGCAAGCCCGGCCGGGCCTACGGCTTGGGGCGGCCGGGCCCGGCGGCGGGCTGTGTCCG
CGGGGAGCGTCCAGGCTGGGCCGCAGGACCGGGCGCGGAGCCTCGCAGGGTCGGGCTCGGG
CTCCCTAACCGTCTCCGCTTCTTCCGCCAGTCGGTGGCCGGGCTGGCGGCGCGGTTGCAGCGGC
AGTTCGTGGTGCGGGCCTGGGGCTGCGCGGGCCCTTGCGGCCGGGCAGTCTTTCTGGCCTTCG
GGCTAGGGCTGGGCCTCATCGAGGAAAAACAGGCGGAGAGCCGGCGGGCGGTCTCGGCCTGT
CAGGAGATCCAGGCAATTTTTACCCAGAAAAGCAAGCCGGGGCCTGACCCGTTGGACACGAGA
CGCTTGCAGGGCTTTCGGCTGGAGGAGTATCTGATAGGGCAGTCCATTGGTAAGGGCTGCAGT
GCTGCTGTGTATGAAGCCACCATGCCTACATTGCCCCAGAACCTGGAGGTGACAAAGAGCACC
GGGTTGCTTCCAGGGAGAGGCCCAGGTACCAGTGCACCAGGAGAAGGGCAGGAGCGAGCTCC
GGGGGCCCCTGCCTTCCCCTTGGCCATCAAGATGATGTGGAACATCTCGGCAGGTTCCTCCAGC
GAAGCCATCTTGAACACAATGAGCCAGGAGCTGGTCCCAGCGAGCCGAGTGGCCTTGGCTGGG
GAGTATGGAGCAGTCACTTACAGAAAATCCAAGAGAGGTCCCAAGCAACTAGCCCCTCACCCCA
ACATCATCCGGGTTCTCCGCGCCTTCACCTCTTCCGTGCCGCTGCTGCCAGGGGCCCTGGTCGAC
TACCCTGATGTGCTGCCCTCACGCCTCCACCCTGAAGGCCTGGGCCATGGCCGGACGCTGTTCC
TCGTTATGAAGAACTATCCCTGTACCCTGCGCCAGTACCTTTGTGTGAACACACCCAGCCCCCGC
CTCGCCGCCATGATGCTGCTGCAGCTGCTGGAAGGCGTGGACCATCTGGTTCAACAGGGCATC
GCGCACAGAGACCTGAAATCCGACAACATCCTTGTGGAGCTGGACCCAGACGGCTGCCCCTGG
CTGGTGATCGCAGATTTTGGCTGCTGCCTGGCTGATGAGAGCATCGGCCTGCAGTTGCCCTTCA
GCAGCTGGTACGTGGATCGGGGCGGAAACGGCTGTCTGATGGCCCCAGAGGTGTCCACGGCC
CGTCCTGGCCCCAGGGCAGTGATTGACTACAGCAAGGCTGATGCCTGGGCAGTGGGAGCCATC
GCCTATGAAATCTTCGGGCTTGTCAATCCCTTCTACGGCCAGGGCAAGGCCCACCTTGAAAGCC
GCAGCTACCAAGAGGCTCAGCTACCTGCACTGCCCGAGTCAGTGCCTCCAGACGTGAGACAGTT
GGTGAGGGCACTGCTCCAGCGAGAGGCCAGCAAGAGACCATCTGCCCGAGTAGCCGCAAATG
TGCTTCATCTAAGCCTCTGGGGTGAACATATTCTAGCCCTGAAGAATCTGAAGTTAGACAAGAT
GGTTGGCTGGCTCCTCCAACAATCGGCCGCCACTTTGTTGGCCAACAGGCTCACAGAGAAGTGT
TGTGTGGAAACAAAAATGAAGATGCTCTTTCTGGCTAACCTGGAGTGTGAAACGCTCTGCCAGG
CAGCCCTCCTCCTCTGCTCATGGAGGGCAGCCCTG
SEQ ID NO: 2
ATGGCGGTTCGACAAGCTCTTGGAAGAGGCCTGCAACTGGGCAGAGCACTGCTGCTGAGATTC
ACCGGAAAACCCGGCAGAGCCTATGGCCTTGGTAGACCTGGACCTGCCGCCGGATGTGTCAGA
GGCGAAAGACCTGGATGGGCTGCTGGACCTGGTGCCGAGCCTAGAAGAGTTGGACTGGGCCT
GCCTAACCGGCTGCGGTTCTTTAGACAGTCTGTGGCCGGACTGGCCGCCAGACTGCAGAGACA
GTTTGTCGTTAGAGCCTGGGGCTGCGCCGGACCTTGTGGTAGAGCTGTGTTTCTGGCCTTTGGC
CTCGGCCTGGGACTGATCGAAGAGAAGCAGGCCGAAAGCAGACGGGCCGTGTCTGCCTGTCA
AGAGATCCAGGCCATCTTCACCCAGAAGTCCAAGCCTGGACCAGATCCTCTGGACACCAGACGG
CTGCAGGGCTTCAGACTGGAAGAGTACCTGATCGGCCAGAGCATCGGCAAGGGATGTTCTGCC
GCCGTGTACGAGGCCACCATGCCTACACTGCCCCAGAACCTGGAAGTGACCAAGAGCACAGGA
CTGCTGCCTGGCAGAGGCCCTGGAACATCTGCTCCAGGCGAGGGACAAGAAAGAGCACCTGGC
GCTCCAGCCTTTCCACTGGCCATCAAGATGATGTGGAACATCAGCGCCGGCAGCAGCTCTGAGG
CCATCCTGAATACCATGAGCCAAGAGCTGGTGCCCGCCAGCAGAGTTGCTCTGGCTGGCGAAT
ATGGCGCCGTGACCTACCGCAAGTCTAAGAGAGGACCCAAGCAGCTGGCCCCTCATCCTAACAT
CATCCGGGTGCTGAGAGCCTTCACCAGCAGTGTTCCTCTGCTTCCTGGCGCACTGGTGGACTAC
CCTGATGTGCTTCCTAGCAGACTGCACCCTGAAGGACTCGGCCACGGCAGAACCCTGTTCCTCG
TGATGAAGAACTACCCCTGCACACTGCGGCAGTACCTGTGCGTGAACACCCCATCTCCAAGACT
GGCTGCCATGATGCTGCTGCAGCTGCTGGAAGGCGTGGACCATCTGGTGCAGCAGGGAATCGC
CCACAGAGATCTGAAGTCCGACAACATCCTGGTGGAACTGGACCCCGATGGCTGTCCTTGGCTG
GTTATCGCCGATTTCGGCTGCTGTCTGGCCGACGAGTCTATCGGACTCCAGCTGCCTTTCTCCTC
TTGGTACGTGGACAGAGGCGGCAACGGCTGTCTGATGGCTCCCGAAGTGTCTACCGCTAGACC
AGGACCTAGAGCCGTGATCGACTACAGCAAAGCCGATGCCTGGGCCGTGGGAGCCATTGCCTA
TGAGATCTTCGGCCTGGTCAACCCCTTCTACGGACAAGGCAAAGCCCACCTGGAAAGCAGAAG
CTATCAAGAGGCCCAGCTGCCAGCTCTGCCTGAGTCTGTTCCACCTGATGTGCGGCAGCTTGTC
AGGGCTCTGCTGCAGAGAGAGGCCAGCAAAAGACCTTCTGCCAGAGTGGCCGCCAACGTGCTG
CATCTTAGTCTGTGGGGCGAGCACATTCTGGCCCTGAAGAACCTGAAGCTGGACAAGATGGTC
GGATGGCTGCTCCAGCAGTCTGCCGCTACACTGCTCGCCAATAGACTGACCGAGAAGTGCTGC
GTGGAAACGAAGATGAAGATGCTGTTCCTGGCCAACCTGGAATGCGAGACACTGTGTCAGGCC
GCTCTGCTCCTCTGTTCTTGGAGAGCCGCTCTG
SEQ ID NO: 3
ATGGCCGTGCGACAGGCACTGGGCCGGGGCCTGCAGCTGGGGGGGGCCCTCCTGCTACGGTTT
ACCGGCAAGCCTGGCAGAGCCTATGGCCTGGGCAGACCCGGCCCAGCTGCCGGCTGTGTGCGG
GGCGAGCGTCCAGGATGGGCCGCCGGACCCGGGGCAGAGCCACGCAGAGTGGGCCTGGGTCT
GCCCAACAGGCTTAGATTTTTCCGCCAGAGCGTGGCTGGACTGGCTGCCAGGCTGCAGCGGCA
GTTTGTGGTGCGCGCCTGGGGGTGTGCCGGGCCTTGCGGCCGGGCCGTGTTTCTCGCCTTTGG
ACTGGGACTGGGCCTGATTGAAGAAAAGCAGGCCGAGTCCAGAAGGGCCGTCAGCGCTTGCC
AGGAGATCCAGGCCATTTTCACCCAGAAGTCAAAGCCCGGCCCAGATCCACTGGATACCCGAA
GGCTGCAGGGCTTCAGGCTTGAGGAGTACCTGATCGGGCAGAGTATTGGCAAAGGCTGTTCAG
CTGCCGTGTATGAGGCAACAATGCCCACCCTGCCCCAGAATCTGGAGGTGACCAAATCAACAG
GACTGCTGCCAGGGAGAGGGCCAGGAACCAGCGCACCCGGGGAGGGCCAAGAACGGGCCCC
CGGCGCACCAGCCTTCCCTCTGGCAATCAAGATGATGTGGAATATCTCTGCCGGCAGTAGCTCA
GAAGCCATCCTGAATACTATGAGCCAGGAGCTGGTCCCCGCTTCCAGAGTGGCCCTGGCTGGC
GAATACGGCGCTGTGACATACAGGAAGAGTAAAAGGGGCCCAAAGCAGCTGGCCCCTCATCCC
AATATAATCAGAGTGCTGAGGGCCTTTACCTCCAGCGTGCCTTTATTGCCTGGCGCCCTGGTTGA
TTACCCCGACGTGCTGCCCTCACGCCTCCATCCTGAGGGACTGGGACATGGCAGGACCCTGTTT
CTGGTGATGAAGAACTATCCTTGCACCCTGCGGCAGTATTTGTGCGTCAATACTCCATCCCCCCG
GCTGGCCGCCATGATGCTGCTGCAGCTCCTGGAAGGAGTGGATCATCTGGTGCAGCAGGGAAT
CGCCCATAGGGACCTGAAATCCGATAACATCCTGGTGGAACTGGACCCCGACGGATGTCCATG
GCTGGTGATCGCTGACTTCGGATGCTGCCTGGCCGACGAGAGCATCGGCCTGCAGCTGCCTTTC
AGCTCTTGGTATGTAGACAGAGGCGGCAATGGTTGCCTGATGGCCCCTGAAGTGTCTACAGCA
CGGCCCGGACCCAGGGCAGTGATTGATTACTCCAAGGCTGATGCATGGGCCGTGGGCGCAATC
GCCTACGAAATCTTTGGGTTGGTGAACCCATTCTATGGACAGGGCAAAGCCCACCTGGAAAGC
AGGAGTTACCAGGAAGCACAGCTGCCCGCTCTGCCTGAATCCGTGCCACCTGATGTGCGGCAG
CTTGTGCGGGCCCTGCTCCAACGCGAAGCCTCTAAGAGACCATCTGCAAGAGTCGCCGCCAACG
TGCTGCATCTGAGCCTGTGGGGCGAACACATCCTGGCACTGAAGAACCTGAAGCTGGATAAGA
TGGTGGGATGGCTGCTGCAGCAGAGCGCCGCCACCCTGCTGGCCAACCGTCTGACCGAAAAAT
GCTGTGTGGAGACAAAAATGAAGATGCTGTTCCTGGCTAATCTGGAGTGCGAGACACTGTGCC
AGGCCGCCCTGCTGCTGTGCAGCTGGAGGGCCGCCCTC
SEQ ID NO: 9
MAVRQALGRGLQLGRALLLRFTGKPGRAYGLGRPGPAAGCVRGERPGWAAGPGAEPRRVGLGLP
NRLRFFRQSVAGLAARLQRQFVVRAWGCAGPCGRAVFLAFGLGLGLIEEKQAESRRAVSACQEIQA
IFTQKSKPGPDPLDTRRLQGFRLEEYLIGQSIGKGCSAAVYEATMPTLPQNLEVTKSTGLLPGRGPGT
SAPGEGQERAPGAPAFPLAIKMMWNISAGSSSEAILNTMSQELVPASRVALAGEYGAVTYRKSKRG
PKQLAPHPNIIRVLRAFTSSVPLLPGALVDYPDVLPSRLHPEGLGHGRTLFLVMKNYPCTLRQYLCVN
TPSPRLAAMMLLQLLEGVDHLVQQGIAHRDLKSDNILVELDPDGCPWLVIADFGCCLADESIGLQLP
FSSWYVDRGGNGCLMAPEVSTARPGPRAVIDYSKADAWAVGAIAYEIFGLVNPFYGQGKAHLESR
SYQEAQLPALPESVPPDVRQLVRALLQREASKRPSARVAANVLHLSLWGEHILALKNLKLDKMVGW
LLQQSAATLLANRLTEKCCVETKMKMLFLANLECETLCQAALLLCSWRAAL

Adeno-associated virus (AAV) is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Bems, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

As used herein, an “AAV vector” or “rAAV vector” refers to a recombinant vector comprising one or more polynucleotides of interest (or transgenes) that are flanked by AAV terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e. a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

AAV is a replication deficient parvovirus, the single-stranded DNA genome of which is about 4.7 kb in length including two 145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). The sequence of the AAVrh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

AAV DNA in the rAAV genomes may be from any AAV variant or serotype for which a recombinant virus can be derived including, but not limited to, AAV variants or serotypes AAV-1, AAV-2, AAV-3, AAV-4, AAV-5, AAV-6, AAV-7, AAV-8, AAV-9, AAV-10, AAV-11, AAV-12, AAV-13 and AAVrh10. Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promote efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV genome, the two complementary halves of scAAV will associate to form one double stranded DNA (dsDNA) unit that is ready for immediate replication and transcription. It will be understood that instead of the full coding capacity found in rAAV (4.7-6 kb), rAAV comprising a self-complementary genome can only hold about half of that amount (=2.4 kb).

Full-length sequences and sequences for capsid genes for most known AAVs are provided in U.S. Pat. No. 8,524,446, which is incorporated herein in its entirety.

AAV vectors may comprise wild-type AAV sequence or they may comprise one or more modifications to a wild-type AAV sequence. In certain embodiments, an AAV vector comprises one or more amino acid modifications, e.g., substitutions, deletions, or insertions, within a capsid protein, e.g., VP1, VP2 and/or VP3. In particular embodiments, the modification provides for reduced immunogenicity when the AAV vector is provided to a subject.

In accordance with the present disclosure, the AAV used herein is a wild-type AAV or a modified AAV. In some embodiments, the AAV used herein comprises a capsid protein having at least 95% identity to wild-type VP1, VP2, or VP3 capsid protein. In some embodiments, the AAV used herein is AAV1.

In accordance with the present disclosure, the polynucleotide encoding PINK1 is operably linked to a promoter. In some embodiments, the promoter is an eukaryotically active promoter, which is capable of promoting initiation of RNA transcription from a polynucleotide in a eukaryotic cell. In some embodiments, the promoter is a tissue-specific promoter, such as a promoter capable of driving expression in a neuron to a greater extent than in a non-neuronal cell. Suitable neuron-specific promoters, include, without limitation, hSYNI (human synapsin), INA (alpha-internexin), NES (nestin), hTH (human tyrosine hydroxylase), FOXA2 (Forkhead box A2), CaMKII (calmodulin-dependent protein kinase II), Prnp (prion protein promoter), Hb9, and NSE (neuron-specific enolase) promoters. In some cases, the promoter is a ubiquitous promoter. A “ubiquitous promoter” refers to a promoter that is not tissue-specific under experimental or clinical conditions. Suitable ubiquitous promoter used herein include, without limitation, CMV, CAG, UBC, PGK, EFI-alpha, EFS (human eukaryotic translation elongation factor 1 α1 short form), SFFV (spleen focus-forming virus promoter), GAPDH, SV40, HBV, chicken beta-actin, and human beta-actin promoters.

In some embodiments, the polynucleotide encoding PINK1 is operatively linked to a hSYNI promoter (SEQ ID NO: 4).

SEQ ID NO: 4
CTGCAGAGGGCCCTGCGTATGAGTGCAAGTGGGTTTTAGGACCAGGATG
AGGCGGGGTGGGGGTGCCTACCTGACGACCGACCCCGACCCACTGGACA
AGCACCCAACCCCCATTCCCCAAATTGCGCATCCCCTATCAGAGAGGGG
GAGGGGAAACAGGATGCGGCGAGGCGCGTGCGCACTGCCAGCTTCAGCA
CCGCGGACAGTGCCTTCGCCCCCGCCTGGCGGCGCGCGCCACCGCCGCC
TCAGCACTGAAGGCGCGCTGACGTCACTCGCCGGTCCCCCGCAAACTCC
CCTTCCCGGCCACCTTGGTCGCGTCCGCGCCGCCGCCGGCCCAGCCGGA
CCGCACCACGCGAGGCGCGAGATAGGGGGGCACGGGCGCGACCATCTGC
GCTGCGGCGCCGGCGACTCAGCGCTGCCTCAGTCTGCGGTGGGCAGCGG
AGGAGTCGTGTCGTGCCTGAGAGCGCAG

In some embodiments, the polynucleotide encoding PINK1 is operatively linked to a CAG promoter (SEQ ID NO: 5).

SEQ ID NO: 5
GACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCCATTGACGT
CAATGGGTGGAGTATTTACGGTAAACTGCCCACTTGGCAGTACATCAAGTGTATCATATGCCAA
GTACGCCCCCTATTGACGTCAATGACGGTAAATGGCCCGCCTGGCATTATGCCCAGTACATGAC
CTTATGGGACTTTCCTACTTGGCAGTACATCTACGTATTAGTCATCGCTATTACCATGGTCGAGG
TGAGCCCCACGTTCTGCTTCACTCTCCCCATCTCCCCCCCCTCCCCACCCCCAATTTTGTATTTATT
TATTTTTTAATTATTTTGTGCAGCGATGGGGGGGGGGGGGGGGGGGGGGGGCGCGCCAGGCG
GGGCGGGGCGGGGCGAGGGGGGGGCGGGGCGAGGCGGAGAGGTGCGGCGGCAGCCAATC
AGAGCGGCGCGCTCCGAAAGTTTCCTTTTATGGCGAGGCGGCGGCGGCGGCGGCCCTATAAAA
AGCGAAGCGCGCGGCGGGCGGGAGTCGCTGCGCGCTGCCTTCGCCCCGTGCCCCGCTCCGCCG
CCGCCTCGCGCCGCCCGCCCCGGCTCTGACTGACCGCGTTACTCCCACAGGTGAGCGGGCGGG
ACGGCCCTTCTCCTCCGGGCTGTAATTAGCGCTTGGTTTAATGACGGCTTGTTTCTTTTCTGTGG
CTGCGTGAAAGCCTTGAGGGGCTCCGGGAGGGCCCTTTGTGCGGGGGGAGCGGCTCGGGGGG
TGCGTGCGTGTGTGTGTGCGTGGGGAGCGCCGCGTGCGGCTCCGCGCTGCCCGGCGGCTGTG
AGCGCTGCGGGCGCGGCGCGGGGCTTTGTGCGCTCCGCAGTGTGCGCGAGGGGAGCGCGGCC
GGGGGCGGTGCCCCGCGGTGCGGGGGGGGCTGCGAGGGGAACAAAGGCTGCGTGCGGGGT
GTGTGCGTGGGGGGGTGAGCAGGGGGTGTGGGCGCGTCGGTCGGGCTGCAACCCCCCCTGCA
CCCCCCTCCCCGAGTTGCTGAGCACGGCCCGGCTTCGGGTGCGGGGCTCCGTACGGGGCGTGG
CGCGGGGCTCGCCGTGCCGGGGGGGGGGGGCGGCAGGTGGGGGTGCCGGGCGGGGGGGG
GCCGCCTCGGGCCGGGGAGGGCTCGGGGGAGGGGCGCGGCGGCCCCCGGAGCGCCGGCGGC
TGTCGAGGCGCGGCGAGCCGCAGCCATTGCCTTTTATGGTAATCGTGCGAGAGGGCGCAGGGA
CTTCCTTTGTCCCAAATCTGTGCGGAGCCGAAATCTGGGAGGCGCCGCCGCACCCCCTCTAGCG
GGCGCGGGGCGAAGCGGTGCGGCGCCGGCAGGAAGGAAATGGGGGGGAGGGCCTTCGTGC
GTCGCCGCGCCGCCGTCCCCTTCTCCCTCTCCAGCCTCGGGGCTGTCCGCGGGGGGACGGCTGC
CTTCGGGGGGGACGGGGCAGGGGGGGGTTCGGCTTCTGGCGTGTGACCGGCGGCTCTAGAGC
CTCTGCTAACCATGTTCATGCCTTCTTCTTTTTCCTACAGCTCCTGGGCAACGTGCTGGTTATTGT
GCTGTCTCATCATTTTGGCAAAGAATTC

The recombinant gene therapy vector disclosed herein may further comprise one or more regulatory elements, such as, an enhancer, an intron, a polyA signal sequence, and a transcript stabilizing element.

In some embodiments, the transcript stabilizing element may be a WPRE (Woodchuck hepatitis virus posttranscriptional regulatory element) sequence, a HPRE (Hepatitis posttranscriptional regulatory element) sequence, a scaffold-attachment region, a 3′ UTR, or a 5′ UTR. In some embodiments, the recombinant gene therapy vector disclosed herein comprises both a 5′ UTR and a 3′ UTR.

Suitable enhancers used herein include, without limitation, a CMV enhancer, a GAPDH enhancer, a β-actin enhancer, or an EF1-α enhancer.

In some embodiments, the recombinant gene therapy vector disclosed herein comprises one or more intron sequences. For example, the intron sequence may be selected from a rabbit globin intron sequence, a chicken β-actin intron sequence, a synthetic intron sequence, or an EF1-α intron sequence.

In some embodiments, the recombinant gene therapy vector disclosed herein comprises a polyA signal sequence. For example, the polyA signal sequence is a rabbit globin polyA signal sequence, a human growth hormone polyA signal sequence, a bovine growth hormone polyA signal sequence, a PGK polyA signal sequence, an SV40 polyA signal sequence, or a TK polyA signal sequence. In some embodiments, the polyA signal sequence may be a bovine growth hormone polyA signal sequence.

In some embodiments of the recombinant gene therapy vector disclosed herein, the expression cassette may comprise an additional polynucleotide encoding a protein other than PINK1 and the PINK1 encoding polynucleotide and the additional polynucleotide are linked by a linker. Suitable linkers include, without limitation, an IRES (internal ribosome entry site) and a 2A peptide encoding sequence.

In various embodiments of the recombinant gene therapy vector disclosed herein, the expression cassette comprises, in 5′ to 3′ order:
CAG promoter and polynucleotide encoding PINK1;
CAG promoter, polynucleotide encoding PINK1, and WPRE;
hTH promoter and polynucleotide encoding PINK1;
EF1a promoter and polynucleotide encoding PINK1;
EF1a promoter, polynucleotide encoding PINK1, and WPRE;
CBA promoter and polynucleotide encoding PINK1; or
CBA promoter, polynucleotide encoding PINK1, and WPRE.

Also within the scope of the present disclosure, the recombinant gene therapy vector comprises a variant polynucleotide encoding PINK1, in which a higher PINK1 expression level is exhibited. That is, a cell transduced with the vector expresses PINK1 at a higher level in compared to the level of PINK1 expressed in a cell of the same type that is transduced with a vector of the same type comprising a wildtype polynucleotide encoding PINK1.

In a further aspect, the present disclosure provides a host cell comprising any of the recombinant gene therapy vectors as provided above. Exemplary host cells include, without limitation, HEK293, 293T, HeLa, Vero, and Sf9 cells.

In a yet further aspect, the present disclosure provides a variant polynucleotide encoding PINK1 and has a sequence of SEQ ID NO: 2 or 3 or has at least about 70%, about 75%, 80%, about 85%, about 90%, about 95%, about 96%, about 97%, about 98%, or about 99% identity to SEQ ID NO: 2 or 3. In some embodiments, the variant polynucleotide has at least 90% identity to SEQ ID NO: 2 or 3. In some embodiments, the variant polynucleotide has at least 95% identity to SEQ ID NO: 2 or 3. In some embodiments, the variant polynucleotide has at least 98% identity to SEQ ID NO: 2 or 3. In some embodiments, the variant polynucleotide has at least 99% identity to SEQ ID NO: 2 or 3.

In a yet further aspect, the present disclosure provides a plasmid comprising the variant polynucleotide encoding PINK1, as described above.

In a yet further aspect, the present disclosure provides a method for increasing expression of PINK1 in cells (e.g., neurons) in a subject by contacting the cells with the recombinant gene therapy vector, as provided above. The method may be practiced in vitro or in vivo, e.g., in a subject in need thereof. In some embodiments, the subject is a human subject. In some embodiments, the human subject is suffering or at risk of developing PD. In some embodiments, the PD is an early-onset PD. In some embodiments, the PD is an early-onset autosomal recessive PD. In some embodiments, the human subject comprises a mutation in PINK1 gene. In some embodiments, the neuron is a primary tyrosine hydroxylase positive neuron. As demonstrated in Example 2, when gene therapy vectors containing PINK1 gene (wild type or variants) were transduced into PINK1 knock-out (KO) HEK293T cells, both PINK1 protein and mRNA expression were detected. In addition, comparing to cells transduced with vectors containing WT PINK1 gene (SEQ ID NO: 1), cells transduced with vectors containing PINK1 variant GT69 (SEQ ID NO: 2) or GT74 (SEQ ID NO: 3) exhibit higher protein and mRNA expression level of PINK1. Moreover, in Example 3, high PINK1 functionality were detected in PINK1 KO HEK293T cells that are transduced with vectors containing PINK1 variant GT69 or GT74.

In a yet further aspect, the present disclosure provides a method for increasing expression of PINK1 in cells in a subject. The method comprises contacting the cells with a recombinant gene therapy vector comprising a variant polynucleotide encoding PINK1, as described above, wherein expression of PINK1 in the cells is increased relative to expression of PINK1 in cells of the same type which are contacted under the same conditions with the same amount of a vector of the same type comprising a wildtype polynucleotide encoding PINK1.

In a yet further aspect, the present disclosure provides a method for inhibiting, reducing, or delaying degeneration or death of neurons by contacting the neurons with the recombinant gene therapy vector, as provided above. The method may be practiced in vitro or in vivo, e.g., in a subject in need thereof. In some embodiments, the subject is a human subject. In some embodiments, the human subject is suffering or at risk of developing PD. In some embodiments, the PD is an early-onset PD. In some embodiments, the PD is an early-onset autosomal recessive PD. In some embodiments, the human subject comprises a mutation in PINK1 gene. In some embodiments, the neuron is a primary tyrosine hydroxylase positive neuron. In some embodiments, MPP+ (1-methyl-4-phenylpyridinium)-induced neurodegeneration is attenuated. In some embodiments, MPTP (1-methyl-4-phenyl-1,2,3,6-tetrahydropyridine)-induced neuron loss is prevented. As demonstrated in Examples 4-5, cortical neurons transduced with vectors containing PINK1 variants (GT69 or GT74) exhibits reduced level of LDH release compared to un-transduced cells.

Administration of the recombinant gene therapy vector or compositions comprising the recombinant gene therapy vector may be by routes standard in the art including, but not limited to, systemic, local, direct injection, parenteral, intravenous, cerebral, cerebrospinal, intrathecal, intracistemal, intraputaminal, intrahippocampal, intranigral, intra-striatal, or intra-cerebroventricular administration. In some cases, administration comprises intravenous, cerebral, cerebrospinal, intrathecal, intracistemal, intraputaminal, intrahippocampal, intra-striatal, or intra-cerebroventricular injection.

In some embodiments, the disclosure provides for local administration and systemic administration of a therapeutically effective amount of the recombinant gene therapy vector disclosed herein. For example, systemic administration may be administration into the circulatory system so that the entire body is affected. Systemic administration includes enteral administration such as absorption through the gastrointestinal tract and parental administration through injection, infusion or implantation.

In some embodiments, actual administration of the recombinant gene therapy vector disclosed herein may be accomplished by using any physical method that will transport the vector into the target tissue of a subject. Suitable administration routes include, but are not limited to, injection into the central nervous system (CNS) or cerebrospinal fluid (CSF) and/or directly into the brain.

Capsid proteins of a recombinant gene therapy vector may be modified so that the vector is targeted to a particular target tissue of interest such as neurons or more particularly a dopaminergic neuron. See, for example, Albert et al. AAV Vector-Mediated Gene Delivery to Substantia Nigra Dopamine Neurons: Implications for Gene Therapy and Disease Models. Genes. 2017 Feb. 8; see also U.S. Pat. No. 6,180,613 and U.S. Patent Pub. No. US20120082650A1, the disclosures of both of which are incorporated by reference herein. In some embodiments, the recombinant gene therapy vector is directly injected into the substantia nigra of the subject.

For purposes of administration, e.g., by injection, various solutions can be employed, such as sterile aqueous solutions. Such aqueous solutions can be buffered, if desired, and the liquid diluent first rendered isotonic with saline or glucose. Solutions of vector as a free acid (DNA contains acidic phosphate groups) or a pharmacologically acceptable salt can be prepared in water suitably mixed with a surfactant such as hydroxpropylcellulose. A dispersion of the vector can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include, but are not limited to, sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating recombinant gene therapy vector in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

Transduction with the recombinant gene therapy vector may also be carried out in vitro. In one embodiment, desired target cells are removed from the subject, transduced with the recombinant gene therapy vector and reintroduced into the subject.

Suitable methods for the transduction and reintroduction of transduced cells into a subject are known in the art. In one embodiment, cells can be transduced in vitro by combining the recombinant gene therapy vector with cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by systemic, local, direct injection, parenteral, intravenous, cerebral, cerebrospinal, intrathecal, intracisternal, intraputaminal, intranigral, intrahippocampal, intra-striatal, or intra-cerebroventricular administration. In some cases, administration comprises intravenous, cerebral, cerebrospinal, intrathecal, intracistemal, intraputaminal, intrahippocampal, intra-striatal, or intra-cerebroventricular injection. Administration may be performed by intrathecal injection with Threndelenburg tilting.

Transduction of cells with the recombinant gene therapy vector disclosed herein results in sustained or increased expression of PINK1. Thus provided herein are methods of administering or delivering recombinant gene therapy vectors which express PINK1 to a mammalian subject, preferably a human being. These methods include transducing tissues (including, but not limited to, the tissues of the brain) with the recombinant gene therapy vector disclosed herein. Transduction may be carried out with gene expression cassettes comprising tissue specific control elements. For example, in one embodiment, transducing neuronal cells and brain tissues is directed by neuron-specific control elements, including, but not limited to, those derived from neuron-enriched promoters, and other control elements.

In a yet further aspect, the present disclosure provides recombinant gene therapy vector comprising a variant polynucleotide encoding PINK1, wherein a cell transduced with the vector expresses PINK1 at a higher level in compared to the level of PINK1 expressed in a cell of the same type that is transduced with a vector of the same type comprising a wildtype polynucleotide encoding PINK1.

In some embodiments, the cells are contacted with the recombinant gene therapy vector comprising the variant polynucleotide encoding PINK1, thereby increasing the expression of PINK1 in the cells relative to expression of PINK1 in cells of the same type which are contacted under the same conditions with the same amount of a vector of the same type comprising a wildtype polynucleotide encoding PINK1.

In some embodiments, the neurons of a subject are contacted with the recombinant gene therapy vectors comprising the variant polynucleotide encoding PINK1, thereby increasing the expression of PINK1 in the neurons relative to expression of PINK1 in neurons of the same type which are contacted under the same conditions with the same amount of a vector of the same type comprising a wildtype polynucleotide encoding PINK1 and thereby inhibiting, reducing, or delaying dysfunction/degeneration or death of neurons in the subject.

In some embodiments, the variant polynucleotide and/or the wildtype polynucleotide is operatively linked to a promoter.

In some embodiments, the vectors are adeno-associated virus (AAV).

In some embodiments, PINK1 is expressed at least about 1.1, 1.2, 1.3, 1.4, 1.5, 1.6, 1.7, 1.8, 1.9, or 2-fold higher by cells or neurons contacted or transduced with the vector comprising the variant polynucleotide encoding PINK1 compared to cells or neurons contacted or transduced with the vector comprising the wildtype polynucleotide encoding PINK1.

In some embodiments, the variant polynucleotide encoding PINK1 has at least about 70%, at least about 80%, at least about 85%, or at least about 90% identity to SEQ ID NO: 2 or SEQ ID NO: 3.

In some embodiments, the cells are neurons, which may be selected from cortical neurons, ependymal cells, glutamatergic neurons, gabaergic neurons, dopaminergic neurons, oligodendrocytes, astrocytes, and microglial cells.

In a yet further aspect, the present disclosure provides a method for inhibiting, reducing, or delaying degeneration or death of neurons in a subject, the method comprising contacting the neurons with an amount of recombinant gene therapy vector comprising a polynucleotide encoding PINK1, wherein the amount of vector is an amount effective to inhibit, reduce, or delay degeneration or death of neurons in the subject.

In some embodiments, the neurons are contacted with an amount of vector corresponding to a multiplicity of infection (MOI) of between about 1 and about 10\hat{ }5, or between about 10 and about 10\hat{ }4, or about 1, or about 10, or about 10\hat{ }1, or about 10\hat{ }2, or about 10\hat{ }3, or about 10\hat{ }4, or about 10\hat{ }5.

In some embodiments, the polynucleotide is operatively linked to a promoter.

In some embodiments, the polynucleotide is a variant polynucleotide encoding PINK1. And the variant polynucleotide encoding PINK1 has at least about 70%, at least about 80%, at least about 85%, or at least about 90% identity to SEQ ID NO: 2 or SEQ ID NO: 3.

In some embodiments, the vectors are adeno-associated virus (AAV).

In some embodiments, the cells are neurons. And the neurons may be selected from cortical neurons, ependymal cells, glutamatergic neurons, gabaergic neurons, dopaminergic neurons, oligodendrocytes, astrocytes, and microglial cells.

### EXAMPLES

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description. And it is well established that the subject matter described and claimed herein is to be viewed and interpreted at the time of filing this patent application.

### Example 1: Design of Human PINK1 Gene Variants

Sixteen (16) human PINK1 gene variants were designed using various design algorithm (see Table 1) and cloned into AAV1 vector (with GT0065 being wildtype PINK1 (WT PINK1)).

TABLE 1

| | | % identity to | |
|---|---|---|---|
| Vector ID | SEQ ID NO | WT PINK1 | % GC content |
| GT0065 | 1 | 100% | 63 |
| GT0066 | — | 80% | 63 |
| GT0067 | 6 | 80% | 63 |
| GT0068 | — | 79% | 65 |
| GT0069 | 2 | 78% | 59 |
| GT0070 | — | 76% | 56 |
| GT0071 | — | 76% | 56 |
| GT0072 | — | 77% | 60 |
| GT0073 | — | 77% | 60 |
| GT0074 | 3 | 78% | 61 |
| GT0075 | — | 82% | 73 |
| GT0076 | — | 81% | 68 |
| GT0077 | 7 | 78% | 63 |
| GT0078 | — | 74% | 55 |
| GT0079 | 8 | 74% | 55 |
| GT0080 | — | 75% | 55 |
| GT0081 | — | 75% | 54 |

SEQ ID NO: 6
atggccgtgcggcaggccctgggccggggcctgcagctgggcagagccctgctgctgcggttcaccggcaaacctggcagag
cctacggcctgggcagacctggcccagccgccggctgcgtgaggggagaaagacccggatgggctgccggcccaggagccg
agcctagaagggtgggcctgggcctgcctaacagactgcggttcttccggcaatccgtggccggactcgccgctagactgcag
agacaatttgtggtgcgggcctggggatgcgccggcccttgcggccgggccgtgttcctggcttttggcctgggactgggcctg
atcgaggaaaagcaggcagaatcccgcagagccgtgtctgcttgccaggagatccaggccatcttcacccagaagagcaaa
cctggccccgatcctctggacaccagacggctgcagggcttcagactggaagagtacctgattggacagtctatcggcaagg
gctgtagcgccgctgtctatgaggccacgatgcctacactgccacagaacctggaagtgacaaagagcaccggcctgctgcc
cggaagaggtcctggaacatctgctcctggcgagggacaggagagagcccctggcgcccctgctttccctctggctatcaaga
tgatgtggaacatcagcgccggctcctcttctgaagccatcctgaacacaatgagccaggagctggtgcccgccagcagggtt
gccctggctggagagtacggcgctgtgacatacagaaagtccaagcggggacccaagcagctggccccccaccctaatatca
tcagagtgctgagagccttcaccagcagcgtgcctctgctgcctggcgccctggtggactaccctgatgtgctgcctagccggc
tccatcctgaaggcctgggccacggccggaccctgttcctggtgatgaagaactacccctgcaccctcagacagtacctgtgc
gtgaacacccctagccccagactggccgctatgatgctgctgcagctgctggaaggcgtggatcacctggtgcagcagggcat
cgcccacagagatctgaagagcgacaacatcctggtggaacttgaccctgacggctgtccttggctggtaatcgccgacttcg
gctgttgtctggccgacgagagcatcggcctacagctgccattcagctcttggtacgtggacagaggcggcaacggctgcctg
atggcccctgaggtgtccaccgccagacctggccccagagccgtgattgactacagcaaggccgatgcctgggccgtgggcg
ccatcgcctacgagatcttcggcctggtcaaccctttctacggccagggcaaggctcacctggaaagcagaagctaccagga
ggcccagctgcccgccctgcctgagagtgtgccacctgacgtgcgccaactcgtcagagccctgctgcaaagagaggccagc
aaaagacccagcgcccgtgtggccgccaatgtgctgcacctttctctgtggggcgaacacatcctggccctgaaaaacctcaa
gctggacaagatggtgggctggctgctgcagcaaagcgccgcgaccctgctggctaatagactgaccgagaagtgctgcgtg
gaaaccaagatgaaaatgctgtttctggccaacctggagtgcgagacactgtgtcaggcagccctgctgctgtgcagctggcg
ggccgccctt
SEQ ID NO: 7
atggctgtccgccaagccttagggagagggctgcagctgggcagagcgctcttgctgaggttcaccggaaagcccggacgcg
cttacggcctcggaaggccaggtcctgcagcgggttgcgtgcgaggggaacggcctggttgggcagcaggaccaggagccg
agccacggagagtgggactcggcctgcctaaccgcctccgcttcttccggcaaagcgtggccggtcttgccgcacggcttcag
cgccagttcgtggtccgggcttggggttgtgccggaccttgtggacgggccgtgttcctggccttcggactgggcctgggacta
atcgaggaaaagcaggccgaatcccggagagcggtgtcagcgtgccaggaaatccaggccatattcacccagaagtccaag
ccgggaccagacccactggacactaggcggctgcagggcttccggctcgaggaatacctcatcggccagtccattggcaaag
gttgttcggccgccgtgtacgaggccaccatgcccaccctgccgcaaaacctggaagtgaccaagtccactgggcttctgccc
ggaagaggacccgggacttctgccccgggagaaggacaggagcgggctcctggagcacccgcgtttccgctggccattaag
atgatgtggaacatctcggccggttccagctccgaggccatcctgaacacgatgtcccaagagctggtgccggcgtcaagagt
cgccttggctggagaatacggcgccgtgacctaccgcaaatccaagcgcggcccgaaacagctggcgccacaccctaacatc
atcagagtgctgcgcgcgttcacctcctccgtgcccctgcttcctggcgctctggtggactatccggacgtgctcccgtcgagac
tgcaccccgaagggttggggcatggacgcaccctgtttctggtcatgaagaactacccctgcactctgcggcagtatctgtgcg
tcaacaccccgtcacctcgccttgccgcgatgatgctgctccaactgctggaaggagtggaccacttggtgcagcagggtatc
gcccaccgggatctcaagtccgacaacattctggtggagcttgatcccgatggatgcccctggctggtcatcgccgacttcggc
tgctgcctggctgacgaaagcatcggactccagttgccgttctcgtcttggtacgtggatcggggcggaaatggctgtctgatg
gcccccgaagtctccactgcacgccctggacctagggccgtgattgactactcgaaggccgatgcatgggccgtcggagccat
cgcctacgagattttcggcttggtgaacccgttctacggccaagggaaggcccacctggaaagcaggagctaccaggaggcc
caattgcccgcccttcctgagtccgtgccgcctgacgtgcgacagctcgtgcgcgcactcctgcaacgggaggcctcgaagag
gccgtcagctagggtggccgcgaacgtgctgcatctgtcgctgtggggggaacacattctggccctgaagaacctcaagctcg
acaagatggtcggctggctgctgcagcaatccgcggccactctgctcgccaatcggctgaccgagaagtgctgcgtggaaac
caagatgaagatgctgtttctcgccaacctggagtgcgaaacactgtgccaggctgccctgctgctctgctcctggagagccgc
gctg
SEQ ID NO: 8
atggcggtcaggcaagcactgggtcgaggactgcagctcggtcgggctcttcttttgcggtttaccggtaaacccggtcgggc
atacgggctgggacgacccggacctgccgctggatgtgttagaggggagaggccgggttgggcagccggacccggagcag
gccaagaagagtgggtctgggactgcccaaccgactcaggtttttcaggcagtctgtagcagggttggcagctaggctgcaa
agacaattcgtggtacgggcgtggggatgcgccgggccatgtggtcgggcggtctttttggccttcggtctggggctcggtttg
atcgaggagaaacaagctgagtcccgaagagcggtatctgcgtgtcaagagatacaggccatattcacgcaaaaatcaaa?
ccaggtcccgaccctttggacactaggagactgcaaggattccgcctcgaagagtatctgatcggtcaatcaataggaaaag
gttgttcagctgctgtgtatgaagctacgatgcctaccctgcctcaaaatctcgaagtaacgaagtcaaccggattgttgcca
ggcgagggccaggcacttctgcacctggggaagggcaagagagggcaccgggtgcaccggcattccccctcgcaattaaa:
tgatgtggaacatatctgccggctcttccagcgaggcaatattgaacacaatgtctcaggaacttgtgcccgctagtcgcgta;
cacttgccggcgaatatggtgctgtaacttatagaaaaagcaaaagggggccaaagcagctggcgccgcatcccaacatca
aagggtcttgcgagcgttcacttcaagcgtgcctcttcttcctggagcgcttgtcgattatcccgatgtcttgccgtcccgattg(
acccagagggtctcggtcacggacgaacgctgttcttggtgatgaaaaactacccttgtactctgcgacaatacttgtgtgtc.
acacgccctctcctcgattggcagcgatgatgcttctccaacttcttgagggagtggaccatttggtccagcagggaatcgcg(
atcgggaccttaagtcagacaacatattggtagaactcgatccggatggttgcccctggctcgtgatcgcggattttgggtgct
gcttggcagacgaatcaataggactgcaattgccgttttctagttggtatgtcgatagggggggaacgggtgcttgatggca
ccgaagtgtctacggcgcgacccggcccacgagcagttattgattactcaaaggcggatgcctgggctgtcggcgctattgc:
tacgaaatcttcggcctcgtgaaccctttctatgggcaaggaaaagcgcaccttgaatccagaagttaccaagaagcgcagt
gcctgcgctcccagagagcgtacctccagatgttagacagctcgtacgggcactgcttcagagagaggcgagcaaaagacc
atccgcaagggttgccgccaatgtgttgcatctgtccttgtggggagagcatatcttggcccttaagaatctgaagctggataa
aatggtaggatggctcttgcaacaatctgcggcaacgcttctggctaataggctgacagaaaaatgctgcgtagagaccaaa

atgaagatgttgttcctcgccaaccttgagtgcgaaacgctctgtcaagcggcattgctcttgtgtagctggagagcagctctt

### Example 2: Screening for Protein Expression

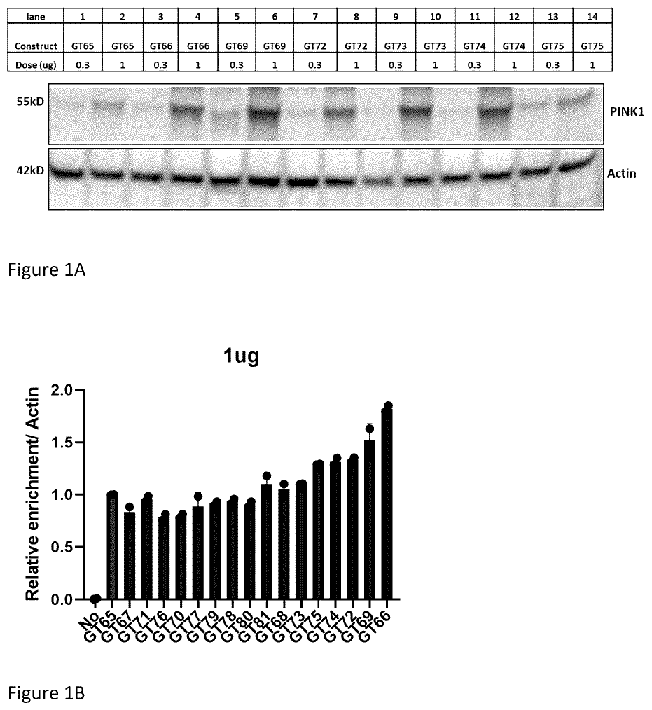

AAV1 CIS plasmids for WT PINK1 (GT65) and all 16 PINK1 variants (GT66-GT81) were generated. The plasmids were then tested in PINK1 knock-out (KO) Human Embryonic Kidney (HEK) 293T cells for protein and mRNA expression. FIG. 1A shows the relative PINK1 protein levels for 6 representative PINK1 variant constructs in PINK1 KO HEK293T cells. The PINK1 KO HEK293T cells were cultured at the same cell densities for 24 hours. The next day these cells were treated with plasmid DNA bearing the 6 different PINK1 variants at a concentration of 0.3 microgram and 1 microgram. The treated cells were incubated for 48 hours and were collected for protein estimation using Western Blot. Specifically, the cells were lysed, and the lysate was subjected to electrophoresis to separate out the proteins by size and charge. The separated proteins were then queried with antibodies specific to PINK1 protein and detected by autoradiography. A housekeeping protein, namely Actin was used as an internal control to normalize for protein levels across the samples. The PINK1 and Actin protein bands were measured for intensity using an imaging software and plotted as shown in FIG. 1B. FIG. 1B shows a representative Western blot analysis of the 16 PINK1 variant constructs that were overexpressed in PINK1 KO HEK293T cells. The graph represents the relative enrichment of PINK1 protein compared to GT65 (WT PINK1) and is plotted in increasing order of expression. The experiment was repeated three times. Statistical analysis was done in GraphPad Prism software (one-way ANOVA, p<0.0001).

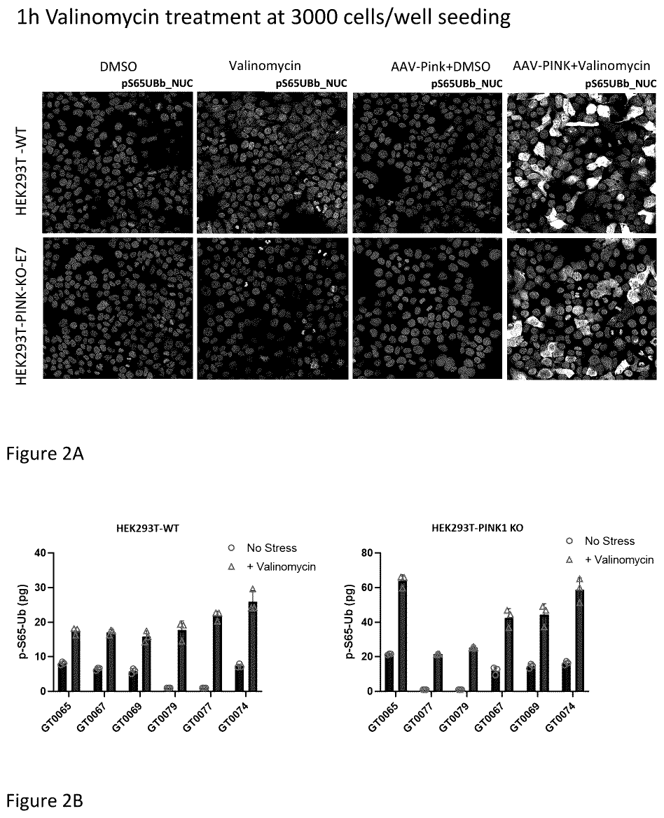

Example 3: Functional Assay of Selected PINK1 variants. This set of data shows that the AAV1 CIS plasmids selected from the expression data above are functional inside the cells that express them. FIG. 2A shows representative images from HEK293T wildtype and PINK1 knockout cells that were treated with DMSO vehicle or Valinomycin drug before and after overexpressing AAV1-PINK1. Valinomycin is a mitophagic stressor that causes PINK1 protein to stabilize on the surface of mitochondria triggering a PINK1 specific modification on another protein called Ubiquitin. This modification, called pS65-Ub can be detected by immunocytochemistry (FIG. 2A) and by Mesoscale discovery (MSD, FIG. 2B) Assay of pS65-Ub and is a direct readout for PINK1 function. Both HEK293T wildtype and KO cells that overexpress AAV1-PINK1 show a very strong pS65-Ub signal after Valinomycin treatment (last panel) compared to DMSO (first and third panel) or background levels (second panel). The overexpression of the PINK1 variants was done as described earlier.

Five (5) selected PINK1 variants (GT67, GT69, GT74, GT77, and GT79), along with WT PINK1 (GT65) were further tested in a PINK1-specific functional assay that measures PINK1 phosphorylation of ubiquitin at serine-65 (p-S65-Ub) after the induction of mitophagic stress with valinomycin. FIG. 2B shows representative data from pS65-Ub MSD assay in PINK1 WT and KO HEK293T overexpressing the 5 PINK1 variants (GT67, GT69, GT74, GT77, and GT79) as compared to the WT PINK1 (GT0065). Blue bars represent the pS65-Ub levels under normal or unstressed conditions while the red bars show the stabilized pS65-Ub levels as a response to PINK1 overexpression and function.

### Example 4: Neuronal Survival Assessments

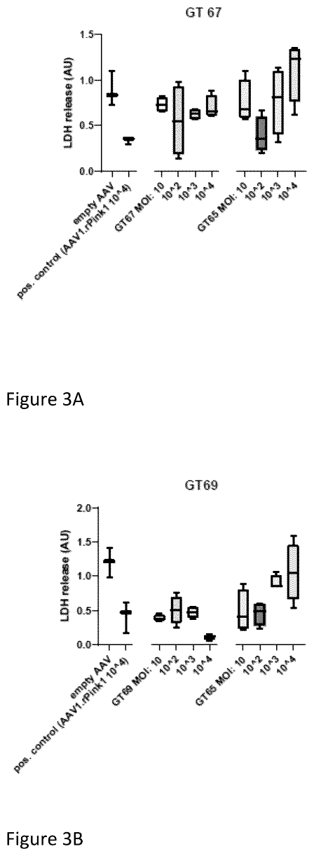

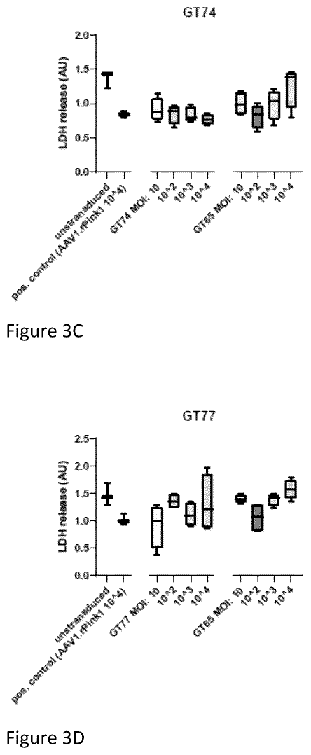

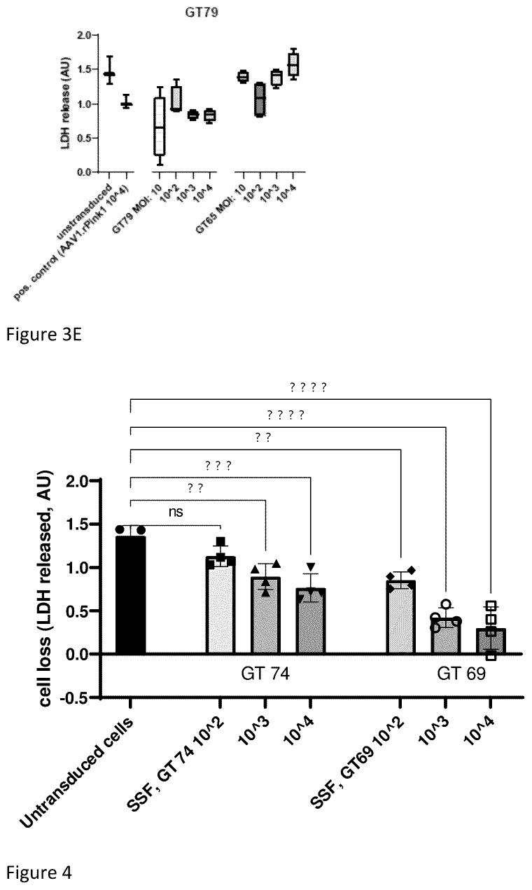

Neurotoxin rescue experiments were conducted in primary neuronal cultures. LDH release assays measure cytotoxicity in cortical neurons transduced with AAV1-PINK1 vectors. Shown here are the amounts of LDH released into media for the vectors containing one of the selected 5 PINK1 variants compared to the WT PINK1 vector (“GT65”) after neuronal cultures were challenged with MPP+ (500 uM) at 7 days post-transduction at indicated MOIs. Embryonic day 18 (E18) rat cortices were dissociated using a standard protocol and plated on PDL coated 96 well plates and incubated at 37° C. for 7 days. The E18 neurons were then transduced with a AAV1 vector containing one of the 5 PINK1 variants or WT PINK1 at Multiplicity of Infection (MOI) rates ranging from 10 to 10\hat{ }4 viral genomes per cell. The cells were incubated for another 7 days and an aliquot of media was collected as pre-treatment control from all wells. The neurotoxin MPP+was added at 500 UM concentration and the cells were incubated with the toxin for 24 hours. The media containing LDH (released due to cell death) was collected post-MPP+treatment and the LDH levels were measured for pre and post treatment conditions using a commercially available colorimetric assay. The graph represents the amount of LDH released from the various treatment conditions as specified on the X axis for GT67 (FIG. 3A), GT69 (FIG. 3B), GT74 (FIG. 3C), GT77 (FIG. 3D) and GT79 (FIG. 3E). AAV1.rPINK1 (rPINK1=Rat PINK1; MOI=10\hat{ }4) is the positive control that shows a rescue of cell death i.e. less LDH released as compared to control (first two bars). In FIGS. 3A and 3B, the controls were rat cortical neurons transduce with empty AAV1 vector, while in FIGS. 3C, 3D, and 3E, the controls were untransduced cortical neurons. Only GT 69 (FIG. 3B) and GT74 (FIG. 3C) showed a similar effect as the positive control at lower MOIs.

### Example 5: Batch-to-Batch Reproducibility of GT69 and GT74 Effects on Neuronal Survival

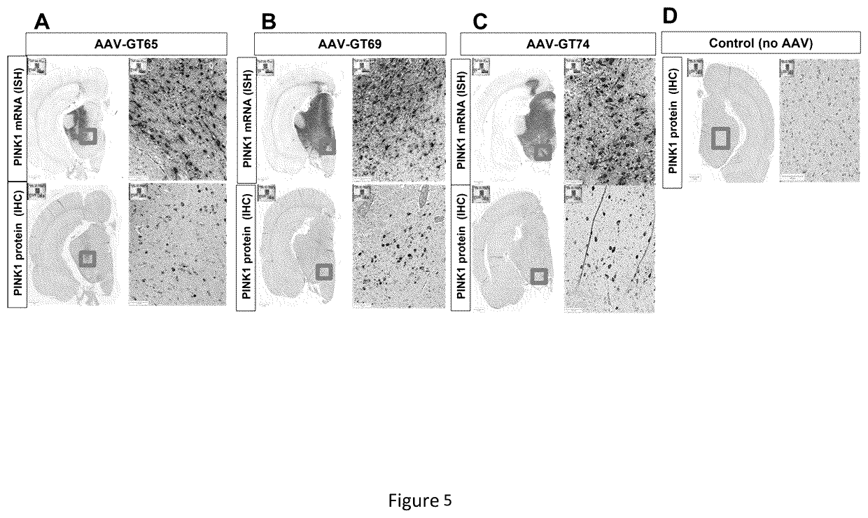

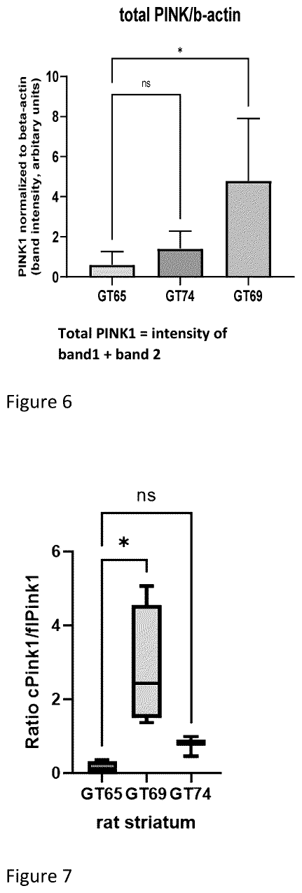

AAV1 vectors containing GT69 and GT74 performed just as well at transducing neurons and attenuating cell death after a toxin challenge, suggesting reproducibility across production sites with these sequences. FIG. 4 shows LDH release assays to measure cytotoxicity as described above in primary cortical neurons transduced with GT69 or GT74 at indicated MOIs. Statistical results from one way ANOVA are indicated with *.

### Example 6: In Vivo PINK1 Expression in Rat

Robust in vivo PINK1 expression is achieved after direct injection into the WT rat brain of AAV encoding all three constructs (FIG. 5). Adult WT rats were stereotaxically injected directly into their midbrains with 6e9 GC of GT65 (A), GT69 (B), or GT74 (C) in a fixed 4 μL volume per injection (n=4 per vector). 3 weeks later, brains were collected and processed for in situ hybridization (ISH) to determine human PINK1 mRNA levels (top images) and immunohistochemistry (bottom images). Representative images of the whole coronal brain slice for the injected hemisphere are shown on the left (scale bar=1 mm), and zoomed in images at the red-outlined boxed portion to the right (scale bar=100 uM). (D) This IHC method has been optimized to detect only human PINK1, so no signal is detected in uninjected WT rats. Additionally, FIGS. 6 and 7 are western blot and qPCR results, respectively, from striatum samples. These show that GT69 and GT74 are as good as or better than GT65 at inducing expression of human PINK1 protein in WT rat striatum.

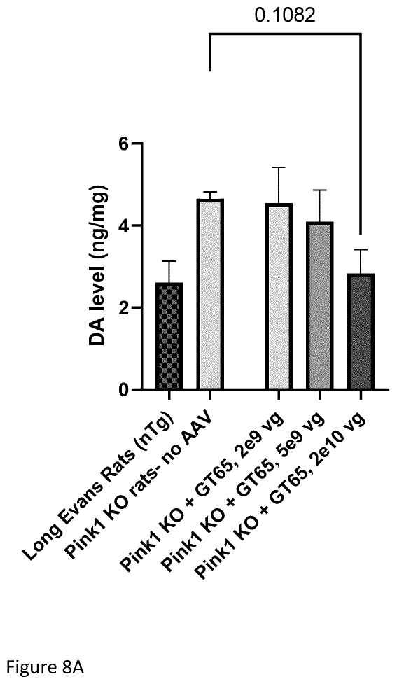

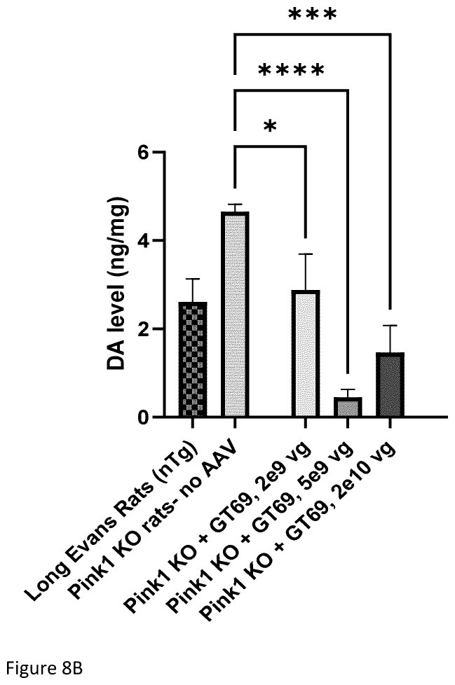

It has been known that at 12 months of age, Pink1 KO mice have increased dopamine (DA) levels in the striatum relative to WT controls (compare checkered versus solid grey bars in FIGS. 8A and 8B). This is thought to be the result of presynaptic compensation resulting from nigrostriatal damage. Treatment with all doses tested of GT69 resulted in dopamine levels that were significantly different than the PINK1 KO controls without any AAV treatment at 6 months post-injection (FIG. 8B), whereas the AAV1.wtPINK1 vector (GT65) at matched dose levels did not significantly lower the increased striatum DA levels closer to WT control levels (FIG. 8A), although there was a nonsignificant trend toward correction at the highest dose tested. These demonstrate that GT69 normalizes dopamine levels in striatal samples from PINK1 KO rats.

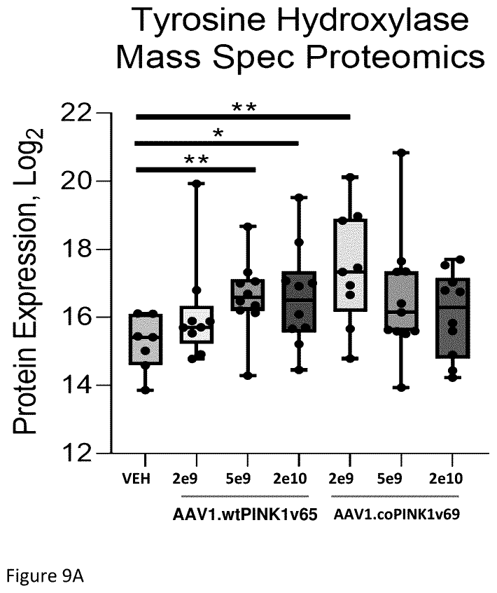

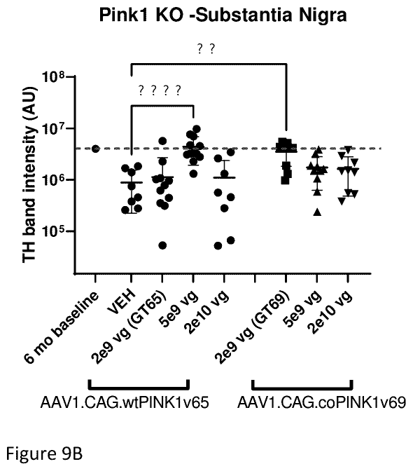

Furthermore, GT69 rescued TH loss at a lower dose than GT65 in PINK1 KO rat midbrain samples. Tyrosine hydroxylase (TH) is a marker of dopaminergic neurons. In Pink1 KO rats at 12 months of age, the minimum effective dose that resulted in a significant increase in midbrain TH levels was 5 e9 vg for GT65, whereas GT69 rescue TH loss at the 2e9 dose. Similar results were obtained by mass spectrometry (FIG. 9A) and western blot (FIG. 9B).

### Example 7: Motor Skills

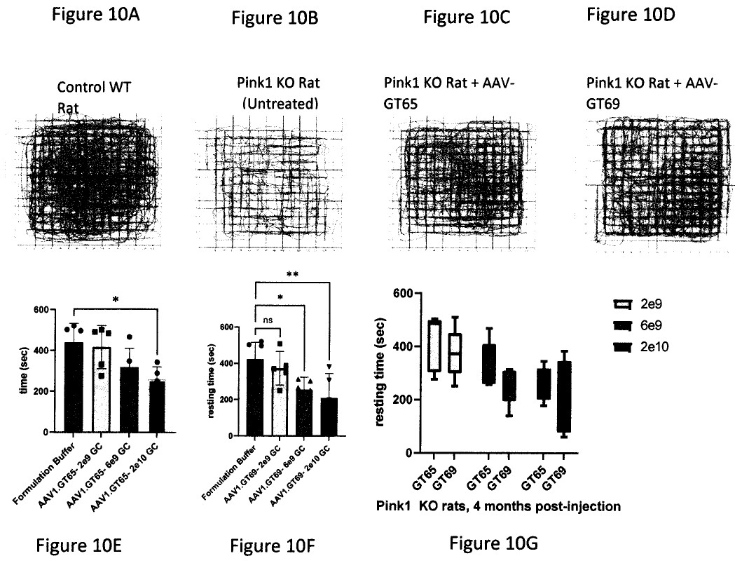

FIGS. 10A-10D show path maps of WT rats, PINK1 KO rats, or PINK1 KO rats transduced with AAV containing WT PINK1 gene (GT65) or variant PINK1 gene (GT69). FIGS. 10E-10G show graphs showing motor skill changes for PINK1 KO rats after transduction with AAV containing WT PINK1 gene (GT65) or variant PINK1 gene (GT69).

### Example 8: In Vivo PINK1 Expression in Canine Brain

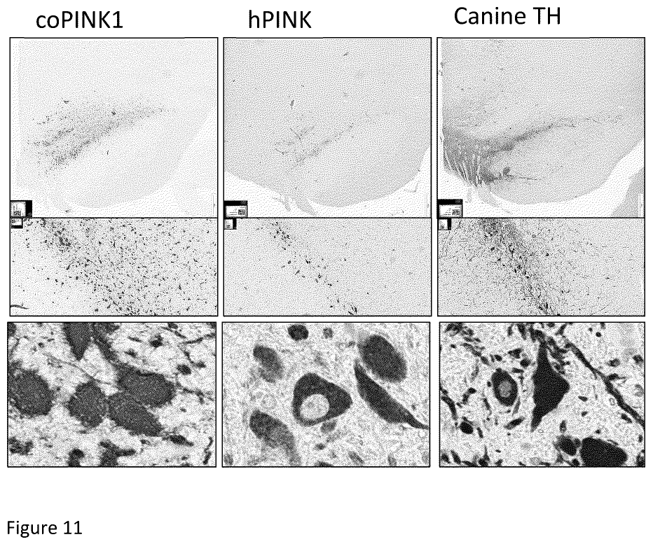

In this example, a single injection of AAV1 vector containing GT69 was given to the substantia nigra of canine brain, which results in robust hPINK1 transgene expression in that region. As shown in FIG. 11, in situ hybridization (ISH) and immunohistochemistry (IHC) for hPINK1 mRNA and protein and canine tyrosine hydroxylase (TH) confirmed desired transduction pattern in the dog brain. These images are representative images of the canine midbrain sections (rep. 1 of 6) at 1-week post-injection with GT69 (1e10 vg in 20 μL).

1. A recombinant gene therapy vector comprising a polynucleotide operatively linked to a promoter, wherein the vector is an adeno-associated virus (AAV) and the polynucleotide encodes PTEN-induced kinase 1 (PINK1) and has at least about 90% identity to SEQ ID NO: 2 or SEQ ID NO: 3.

2. The recombinant gene therapy vector of claim 1, wherein the PINK1 encoding polynucleotide has at least 95% identity to SEQ ID NO: 2 or SEQ ID NO: 3.

3. The recombinant gene therapy vector of claim 1, wherein the PINK1 encoding polynucleotide has at least 98% identity to SEQ ID NO: 2 or SEQ ID NO: 3.

4. The recombinant gene therapy vector of claim 1, wherein the PINK1 encoding polynucleotide has at least 99% identity to SEQ ID NO: 2 or SEQ ID NO: 3.

5. The recombinant gene therapy vector of claim 1, wherein the PINK1 encoding polynucleotide has a sequence of SEQ ID NO: 2 or SEQ ID NO: 3.

6. The gene therapy vector of any one of claims 1-5, wherein the vector is a serotype 1 adeno-associated virus (AAV1).

7. The gene therapy vector of any one of claims 1-6, wherein the promoter is selected from the group consisting of hSYNI (human synapsin), INA (alpha-internexin), NES (nestin), hTH (human tyrosine hydroxylase), FOXA2 (Forkhead box A2), CaMKII (calmodulin-dependent protein kinase II), NSE (neuron-specific enolase), CMV, CAG, UBC, PGK, EFI-alpha, GAPDH, SV40, HBV, chicken beta-actin, and human beta-actin promoters.

8. The gene therapy vector of any one of claims 1-7, which further comprises one or more regulatory elements.

9. The gene therapy vector of claim 8, wherein the one or more regulatory elements are selected from the group consisting of enhancers, introns, poly-A signal sequence, and transcript stabilizing elements.

10. The gene therapy vector of claim 9, wherein the enhancers are selected from the group consisting of CMV enhancer, GAPDH enhancer, β-actin enhancer, and EF1-α enhancer.

11. The gene therapy vector of claim 9, wherein the transcript stabilizing elements are selected from the group consisting of a WPRE (Woodchuck hepatitis virus posttranscriptional regulatory element) sequence, a HPRE (Hepatitis posttranscriptional regulatory element) sequence, a scaffold-attachment region, a 3′ UTR, and a 5′ UTR.

12. The gene therapy vector of any one of claims 1-11, which comprises an expression cassette comprising in 5′ to 3′ order: a) CAG promoter and polynucleotide encoding PINK1; b) CAG promoter, polynucleotide encoding PINK1, and WPRE; c) hTH promoter and polynucleotide encoding PINK1; d) EF1a promoter and polynucleotide encoding PINK1; e) EF1a promoter, polynucleotide encoding PINK1, and WPRE; f) CBA promoter and polynucleotide encoding PINK1; or g) CBA promoter, polynucleotide encoding PINK1, and WPRE.

13. A host cell comprising the gene therapy vector of any one of claims 1-12.

14. The host cell of claim 13, which is selected from the group consisting of HEK293, 293T, HeLa, Vero, and Sf9 cells.

15. A method for increasing expression of PINK1 in cells in a subject by contacting the cells with the recombinant gene therapy vector of any one of claims 1-12.

16. The method of claim 15, wherein the cells are neurons.

17. The method of claim 16, wherein the neurons are primary tyrosine hydroxylase positive neurons.

18. The method of any one of claims 15-17, wherein the subject is a human subject.

19. The method of claim 18, wherein the human subject comprises a mutation in PINK1 gene.

20. The method of claim 18 or 19, wherein the human subject is suffering or at risk of developing Parkinson's Disease (PD).

21. The method of claim 20, wherein the PD is an early-onset PD.

22. The method of claim 20, wherein the PD is an early-onset autosomal recessive PD.

23. A method for inhibiting, reducing, or delaying degeneration or death of neurons of a subject by contacting the neurons with the gene therapy vector of any one of claims 1-12, thereby increasing the expression level of PINK1 in the neurons and inhibiting, reducing, or delaying dysfunction/degeneration or death of the neurons.

24. The method of claim 23, wherein the subject is a human subject.

25. The method of claim 23 or 24, wherein the neurons are a primary tyrosine hydroxylase positive neuron.

26. The method of any one of claims 23-25, wherein MPP+ (1-methyl-4-phenylpyridinium)-induced neurodegeneration is attenuated in the subject.

27. The method of any one of claims 23-25, wherein MPTP (1-methyl-4-phenyl-1,2,3,6-tetrahydropyridine)-induced neuron loss is prevented in the subject.

28. The method of any one of claims 24-27, wherein the human subject comprises a mutation in PINK1 gene.

29. The method of any one of claims 24-28, wherein the human subject is suffering or at risk of developing Parkinson's Disease (PD).

30. The method of claim 29, wherein the PD is an early-onset PD.

31. The method of claim 29, wherein the PD is an early-onset autosomal recessive PD.

32. A polynucleotide encoding PINK1 and has at least about 90% identity to SEQ ID NO: 2 or SEQ ID NO: 3.

33. The polynucleotide of claim 32, wherein the polynucleotide has at least 95% identity to SEQ ID NO: 2 or SEQ ID NO: 3.

34. The polynucleotide of claim 32, wherein the polynucleotide has at least 98% identity to SEQ ID NO: 2 or SEQ ID NO: 3.

35. The polynucleotide of claim 32, wherein the PINK1 encoding polynucleotide has at least 99% identity to SEQ ID NO: 2 or SEQ ID NO: 3.

36. The polynucleotide of claim 32, wherein the PINK1 encoding polynucleotide has a sequence of SEQ ID NO: 2 or SEQ ID NO: 3.

37. A plasmid comprising the polynucleotide of any one of claims 32-36.

38. A recombinant gene therapy vector comprising a variant polynucleotide encoding PINK1, wherein a cell transduced with the vector expresses PINK1 at a higher level in compared to the level of PINK1 expressed in a cell of the same type that is transduced with a vector of the same type comprising a wildtype polynucleotide encoding PINK1.

39. A method for increasing expression of PINK1 in cells in a subject, the method comprising contacting the cells with a recombinant gene therapy vector comprising a variant polynucleotide encoding PINK1, wherein expression of PINK1 in the cells is increased relative to expression of PINK1 in cells of the same type which are contacted under the same conditions with the same amount of a vector of the same type comprising a wildtype polynucleotide encoding PINK1.

40. A method for inhibiting, reducing, or delaying dysfunction/degeneration or death of neurons in a subject, the method comprising contacting the neurons with a recombinant gene therapy vector comprising a variant polynucleotide encoding PINK1, wherein expression of PINK1 in the neurons is increased relative to expression of PINK1 in neurons of the same type which are contacted under the same conditions with the same amount of a vector of the same type comprising a wildtype polynucleotide encoding PINK1.

41. The vector or method of any one of claims 38-40, wherein the variant polynucleotide is operatively linked to a promoter.

42. The vector or method of any one of claims 38-40, wherein each of the variant polynucleotide and the wildtype polynucleotide is operatively linked to a promoter of the same type.

43. The vector or method of any one of claims 38-42, wherein the vector is an adeno-associated virus (AAV).

44. The vector or method of any one of claims 38-43, wherein PINK1 is expressed at least about 1.1, 1.2, 1.3, 1.4, 1.5, 1.6, 1.7, 1.8, 1.9, or 2-fold higher by cells or neurons contacted or transduced with the vector comprising the variant polynucleotide encoding PINK1 compared to cells or neurons contacted or transduced with the vector comprising the wildtype polynucleotide encoding PINK1.

45. The vector or method of any one of claims 38-44, wherein the variant polynucleotide encoding PINK1 has at least about 70%, at least about 80%, at least about 85%, or at least about 90% identity to SEQ ID NO: 2 or SEQ ID NO: 3.

46. The vector or method of any one of claims 38-45, wherein the cell is a neuron.

47. The vector or method of claim 46, wherein the neuron is selected from the group consisting of cortical neurons, ependymal cells, glutamatergic neurons, gabaergic neurons, dopaminergic neurons, oligodendrocytes, astrocytes, and microglial cells.

48. A method for inhibiting, reducing, or delaying degeneration or death of neurons in a subject, the method comprising contacting the neurons with an amount of recombinant gene therapy vector comprising a polynucleotide encoding PINK1, wherein the amount of vector is an amount effective to inhibit, reduce, or delay degeneration or death of neurons in the subject.

49. The method of claim 48, wherein the neurons are contacted with an amount of vector corresponding to a multiplicity of infection (MOI) of between about 1 and about 10\hat{ }5.

50. The method of claim 48, wherein the neurons are contacted with an amount of vector corresponding to a MOI of between about 10 and about 10\hat{ }4.

51. The method of claim 48, wherein the neurons are contacted with an amount of vector corresponding to a MOI of about 1, about 10, about 10\hat{ }1, about 10\hat{ }2, about 10\hat{ }3, about 10\hat{ }4 or about 10\hat{ }5.

52. The method of any one of claims 48-51, wherein the polynucleotide is operatively linked to a promoter.

53. The method of any one of claims 48-51, wherein the polynucleotide is a variant polynucleotide.

54. The method of any one of claims 48-53, wherein the vector is an adeno-associated virus (AAV).

55. The method of any one of claims 48-54, wherein the variant polynucleotide encoding PINK1 has at least about 70%, at least about 80%, at least about 85%, or at least about 90% identity to SEQ ID NO: 2 or SEQ ID NO: 3.

56. The method of claim 56, wherein the neuron is selected from the group consisting of cortical neurons, ependymal cells, glutamatergic neurons, gabaergic neurons, dopaminergic neurons, oligodendrocytes, astrocytes, and microglial cells.
